(19) United States
(12) Patent Application Publication
(10) Pub. No.: US 2011/0287417 A1
(43) Pub. Date: Nov. 24, 2011

(54) Predicting a response to risperidone

(76) Inventor: Maria Arranz, London (GB)

(21) Appl. No.: 12/798,789

(22) Filed: Apr. 9, 2010

Related U.S. Application Data
(63) Continuation of application No. 12/002,839, filed on Dec. 18, 2007, now abandoned.

(30) Foreign Application Priority Data
Dec. 18, 2006 (GB) 0625194.4
Dec. 18, 2006 (GB) 0625194.6
Jul. 9, 2007 (GB) 0713281.4

(51) Int. Cl. C12Q 1/68; C07H 21/04

(52) U.S. Cl. 435/6.11; 536/23.5

(57) ABSTRACT

The invention relates generally to the relative effect of specific genetic polymorphisms in predicting the clinical outcome of risperidone therapy in patients suffering from a psychiatric disease such as schizophrenia.

### TECHNICAL FIELD

The invention relates generally to the relative effect of specific genetic polymorphisms in predicting the clinical outcome of risperidone therapy in patients suffering from a psychiatric disease such as schizophrenia.

### BACKGROUND FIELD

Psychiatric disorders include anxiety disorders, such as obsessive-compulsive disorder, social phobia, or agoraphobia; eating disorders, including anorexia and bulimia; mood disorders, including manic depression (bipolar disorder); cognitive disorders such as dementias; personality disorders and substance abuse-related disorders; and psychotic disorders, such as schizophrenia and delusional disorders. In general, such disorders are amenable to therapy.

However, psychotic patients typically demonstrate varied responses to treatment with pharmaceutical drugs. Consequently, treatment strategies are trial-and-error, which has a negative effect on prognosis and compliance. Methods and products that enable customized drug treatment by identifying genetic components that contribute to the inter-individual differences in drug response and development of drug-induced side effects would improve the quality of care for patients with psychotic diseases significantly.

Typically, response to drug therapy is measured by a scoring system based on scales which assess a variety of symptoms displayed by psychiatric patients. There are many rating scales used for the measuring of the symptoms and severity of disorders in psychiatry. Examples include the Hamilton Depression Rating Scale (Ham-D), Montgomery-øsberg Depression Rating Scale (MADRS), Young Mania Rating Scale, Hamilton Anxiety Rating Scale (Ham-A), Yale-Brown Obsessive-Compulsive Scale (Y-BOCS), Positive and Negative Syndrome Scale (PANSS) Global Assessment of Functioning (GAF) and Clinical Global Impression (CGI) scales.

Of these, PANSS and GAF can be used to assess schizophrenic disorders, and other psychotic conditions.

### Positive and Negative Syndrome Scale (PANSS)

The PANSS originated as a rigorously operationalised method for evaluating positive, negative, and other symptom dimensions in schizophrenia. The PANSS measurement is derived from behavioural information observed during the interview plus a clinical interview and reports by primary care hospital staff or reports by family members.

The ratings provide summary scores on a 7-item positive scale, a 7-item negative scale and a 16-item general psychopathology scale. The added scores provide a PANSS Total Score.

The PANSS ratings should be based on the totality of information pertaining to a specified period, normally identified as the previous week. Each of the 30 items is accompanied by a specific definition as well as detailed anchoring criteria for all seven rating points. These seven points represent increasing levels of psychopathology, as follows: absent; minimal; mild; moderate; moderate severe; severe; extreme. In assigning ratings, a physician first considers whether a symptom is at all present, as judging by the item definition. If the item is present the physician must determine its severity by reference to the particular criteria for the anchoring points. The highest applicable rating point is always assigned, even if the patient meets criteria for lower ratings as well. The rating points minimal to extreme correspond to incremental levels of symptom severity. They are keyed to the prominence of symptoms, their frequency during the observation phase, and above all their disruptive impact on daily living.

### Global Assessment of Functioning (GAF)

The reporting of overall function on Axis V (5) of the Diagnostic and Statistical Manual of Mental Disorders is performed using the Global Assessment of Functioning (GAF) Scale. The GAF scale may be particularly useful in tracking the clinical progress of individuals in global terms, using a single measure. The GAF scale is to be rated with respect only to psychological and occupational functioning.

The present invention is directed to identifying the relative contributions of genetic polymorphism(s) in a gene or a plurality of genes, to the variability displayed by patients in response to treatment with psychotic drugs, in particular risperidone. Risperidone is an antipsychotic drug that is well known to those in the art. It is also known as 4-[2-[4-(6-fluorobenzo[d]isoxazol-3-yl)-1-piperidyl]ethyl]-3-methyl-2,6-diazabicyclo[4.4.0]deca-1,3-dien-5-one. The skilled person appreciates that functional equivalents of risperidone are encompassed by the invention.

The method according to the invention encompasses genes encoding drug-targeted neurotransmitter receptors, transporters and metabolic enzymes. The products and methods encompassed by the genetic polymorphisms identified herein as contributing to the clinical outcome of risperidone therapy can facilitate the improvement of antipsychotic treatment.

### SUMMARY OF THE INVENTION

Risperidone is ineffective, or has sub-optimal efficacy, in treating a significant proportion of individuals with psychotic disorders, and thus a means to determine which patients are likely to benefit from risperidone treatment is desirable. The present inventors have surprisingly discovered that the presence of specific genetic variations (polymorphism(s)) and combinations thereof, is informative regarding the sensitivity of a patient to risperidone. Thus, the present invention provides a method for determining the likelihood that a patient will display a response to treatment with risperidone, based on the patient's genotype.

### Prediction of Response to Risperidone

There is described a method for determining the likelihood of response to risperidone in a patient which comprises assessing the genotype of the patient. In the method according to the invention, genetic polymorphisms are analysed in a plurality of genes comprising one or more genes selected from the group consisting of DRD2, ADRA1A, and 5-HTT, CYP2D6, 5-HT1A, 5-HT2A, DRD4, COMT, NRG1, ChAT, M1 and 5-HT2C.

Particularly useful combinations of genes are set forth below. In further embodiments, algorithms may be used to predict responsiveness based on polymorphisms present in a plurality of genes comprising one or more genes selected from the foregoing group.

In general, it will be recognised by those skilled in the art that the genes and polymorphisms selected, as well as algorithms eventually used to analyse the polymorphisms, can be selected based on the information available on the patient to be tested and the available data concerning association of particular polymorphisms with responsiveness to risperidone. Examples are given below.

### Response to Risperidone Treatment-I

In one embodiment, there is described herein a first example of a method of determining the likelihood of a response to risperidone treatment in a patient by detecting one or more polymorphisms in a plurality of genes comprising one, two or all three of the following genes of the patient: DRD2, ADRA1A, and 5-HTT. In an aspect of this embodiment, the method further comprises detecting one or more polymorphisms in a plurality of genes comprising one, two, three, four or all five of the following genes of the patient: ADRA1A, CYP2D6, 5-HT1A, 5-HT2A and 5-HT2C. This method is effective in predicting responsiveness to risperidone.

In any of these aspects, the polymorphisms detected in ADRA1A include the allelic forms encoding Arg492Cys polymorphism, where there is a genetic variation in the ADRA1A gene which expresses a polypeptide which encodes a cysteine at codon number 492, as opposed to the wild type arginine. Further there is a genetic variation in ADRA1A upstream of the coding region (−6274C/T). In any of these aspects, the polymorphisms detected in DRD2 include the allelic forms of Taq I A2/A1, where there is a genetic variation in the DRD2 gene consisting of a T allele as opposed to the wild type (C). Moreover, in any of these aspects, the polymorphisms detected in 5-HTT include the allelic forms of 2630 T/C. In further embodiments, the polymorphisms detected in CYP2D6 include the allelic forms *4 extensive metabolizer (EM) or poor metabolizer (PM) and/or the polymorphisms detected in 5-HT1A include the allelic forms of −1018-C/G, and/or the polymorphisms detected in 5-HT2A include the allelic forms of −1438-G/A, and/or the polymorphisms detected in 5-HT2C include allelic forms at the loci encoding Cys23Ser.

In a preferred embodiment, the detected allelic forms of the polymorphisms consist of the allelic forms encoding Arg492Cys of ADRA1A, the allelic forms of Taq I A2/A1 of DRD2, and the allelic forms of 2630 T/C in 5-HTT. In another preferred embodiment, the allelic forms of the polymorphisms detected consist of the allelic forms encoding Arg492Cys of ADRA1A, the allelic forms of Taq I A2/A1 of DRD2, the allelic forms of 2630 T/C in 5-HTT, the allelic forms of *4 EM/PM of CYP2D6, the allelic forms of −1018-C/G in 5-HT1A, the allelic forms of −1438-G/A in 5-HT2A, and the allelic forms at the loci encoding Cys23Ser in 5-HT2C.

Variation of the foregoing loci from wild-type is informative of likelihood of response to risperidone treatment in a patient. Generally, at least three polymorphisms in three different genes should be assayed, preferably including at least one polymorphism from the group set forth above. Preferably, at least two polymorphisms from the group set forth above are assayed, and more preferably at least three. However, different polymorphisms in the same genes can be substituted.

### Response to Risperidone Treatment-II

In another embodiment, there is described herein a second example of a method of determining the likelihood of a response to risperidone treatment in a patient by detecting one or more polymorphisms in a plurality of genes comprising one, two or all three of the following genes of the patient: ADRA1A, DRD2 and DRD4

In another embodiment, there is described herein a method of determining the likelihood of a response to risperidone treatment in a patient by detecting one or more polymorphisms in one, two or all three of the following genes of the patient: ADRA1A, DRD2 and DRD4. In an aspect of this embodiment, the method further comprises detecting the allelic forms of one or more polymorphisms in one, two or three of the following genes of the patient: 5-HT1A, CYP2D6, and 5-HT2A. This following method is more consistent than the method of the first example.

In any of these aspects, the polymorphisms detected in ADRA1A include the allelic forms encoding Arg492/Cys, and/or the polymorphisms detected in DRD2 include the allelic forms of Taq I A2/A1, and/or the polymorphisms detected in DRD4 include the allelic forms of −521 C/T. In further embodiments, the polymorphisms detected in 5-HT1A include the allelic forms of −1018 C/G, and/or the polymorphisms detected in CTP2D6 include the allelic forms of *4 A/G (rs3892097), and/or the polymorphisms detected in 5-HT2A include the allelic forms of rs6313 102 T/C.

In a preferred embodiment, the allelic forms of the polymorphisms detected consist of the allelic forms encoding Arg492/Cys of ADRA1A, the allelic forms of Taq I A2/A1 of DRD2, and the allelic forms of −521 C/T in DRD4. In another preferred embodiment, the allelic forms of the polymorphisms detected consist of the allelic forms encoding Arg492/Cys of ADRA1A, the allelic forms of Taq I A2/A1 of DRD2, the allelic forms of −521 C/T in DRD4, the allelic forms of −1018 C/G in 5-HT1A, the allelic forms of *4 extensive metabolizer (EM) or poor metabolizer (PM) in CYP2D6 and allelic forms of 102 T/C in 5-HT2A.

### Algorithms

There are provided algorithms for analyzing the observed genotypic differences, as assayed by detection of polymorphisms. Detecting the polymorphisms preferably further includes determining the copy number of the wild type allele with respect to each polymorphism.

### Improvement of General Response to Risperidone Treatment

General responsiveness to risperidone treatment can be assessed clinically, for example by applying the PANSS Total score and/or the GAF scale. The following algorithms correlate with assessment of effectiveness by measuring general response.

Specifically in one aspect, which corresponds to the first example given above, the likelihood of a response to risperidone treatment (LoR) in said patient can be predicted using the following algorithm: LoR=[1−(−7.432+0.736A1+1.436A2+21.939B1+21.149B2−0.640C1−1.098C2)], where A1=5-HTT 2630-T/T genotype, A2=5-HTT 2630-T/C genotype, B1=D2 Taq I A2/A2 genotype, B2=D2 Taq I A2/A1 genotype, C1=α1A Arg492/Arg492 genotype and C2=α1A Arg492/Cys genotype.

Specifically in another aspect, the likelihood of a response to risperidone treatment (LoR) in said patient can be predicted using the following algorithm: LoR=[1−(+11.853−22.636A1−22.231A2−1.947B1+1.415C1−0.486D1+2.513E1−0.24E2+4.623F1+1.461F2+4.71G1+0.028G2−3.989H1)], wherein A1=5-HTT 2630-T/T genotype, A2=5-HTT 2630-T/C genotype, B1=D2 Taq I A2/A2 genotype, B2=D2 Taq I A2/A1 genotype, C1=α1A Arg492/Arg492 genotype, C2=α1A Arg492/Cys genotype, D1=α1A−6274-C/C genotype, E1=CYP2D6*4 EM/EM genotype, E2=CYPD6*4 EM/PM genotype, F1=5-HT1A−1018-C/C genotype, F2=5-HT1A−1018-C/G genotype, G1=5-HT2A−1438-G/G genotype, G2=5-HT2A−1438-G/A genotype, and H1=5-HT2C Cys23Ser/Cys23Ser, CysSer or Cys23Ser genotypes. The 5-HT2C gene is in the X chromosome; so homozygotes (females) and hemizygotes (males) for Cys23, and heterozygotes Cys23/Ser23 (only females could be heterozygotes) were grouped together for simplicity). The sequences of the polymorphic genotypes in this algorithm are listed in Table 1A below.

TABLE 1A
5-HT1A −1018 G/C GeneID: 3350
Rs6295
(SEQ ID NO: 52)
cgaGAACGGAGGTAGCTTTTTAAAAA[C/G]GAAGACACACTCGGTCTTC
TTCCAT
5-HT2A −1438 G/A GeneID: 3356
Rs6311
(SEQ ID NO: 53)
TATGTCCTCGGAGTGCTGTGAGTGTC[C/T]GGCACTTCCATCCAAAGCC
AACAGT
5-HT2C Cys23Ser GeneID: 3358
Rs6318
(SEQ ID NO: 54)
CCTAATTGGCCTATTGGTTTGGCAAT[C/G]TGATATTTCTGTGAGCCCA
GTAGCA
5-HTT 2630 C/T (SLC6A4) GeneID: 6532
Rs1872924
(SEQ ID NO: 55)
CCAAATGTAGCCACACATCATAGTCA[C/T]CTAgattcctgggtctacc
ccagac
Alpha 1A Arg492Cys (also described as Arg 347Cys)
GeneID: 148
Rs1048101
(SEQ ID NO: 56)
AGAATGTCTTGAGAATCCAGTGTCTC[C/T]GCAGAAAGCAGtCTTCCAA
ACATGC
Alpha 1A −6274 T/C GeneID: 148
rs2019442
(SEQ ID NO: 57)
acatatgaattttggggagaacacaa[A/G]cattcagacaatagcaTAT
ACATAT
CYP2D6*4 Gene ID: 1565
Rs3892097
(SEQ ID NO: 58)
CCCTTACCCGCATCTCCCACCCCCA[A/G]GACGCCCCTTTCGCCCCAAC
GGTCT
DRD2 Taq IA GeneID: 1813
Rs1800497
(SEQ ID NO: 59)
TGGACGTCCAGCTGGGCGCCTGCCT[C/T]GACCAGCACTTTGAGGATGG
CTGTG

In any of the embodied methods described herein, the response is determined to be beneficial, if there is an improvement of 20 points or more in the GAF scales, or at least a 30% decrease in PANSS values after risperidone treatment in the patient.

### —Measured by PANSS

In one aspect, corresponding to the second example given above and which is consistent in predicting effectiveness of risperidone as assessed by PANSS total score, determining the likelihood of general response to risperidone treatment in said patient (LoR) is calculated according to the following algorithm: [1−(−1.565+2.293A1−0.821A2+1.521B1−0.421C1+1.443C2)], where A1=α1A Arg492/Arg492, A2=α1A Arg492/Cys, B1=D2 Taq I A2/A2, C1=D4−521 C/C and C2=D4−521 C/T, as measured by PANSS.

Specifically in another aspect, the likelihood of response to risperidone treatment (LoR) in said patient is calculated according to the following algorithm: [1−(−5.381+2.831A1−0.542A2+1.904B1−0.310C1+2.160C2+22.479D1+1.68D2−19.014E1+0.424E2+1.347F1+2.166F2)], wherein A1=α1A Arg492/Arg492, A2==α1A Arg492/Cys, B1=D2 Taq A2/A2, C1=D4−521 C/C and C2=D4−521 C/T, D1=5-HT1A−1018 C/C, D2=5-HT1A−1018 C/G, E1=CYP2D6*4 A/A, E2=CYP2D6*4 A/G, F1=5-HT2A 102 T/T, and F2=5-HT2A 102 T/C, as measured by PANSS.

### —Measured by GAF

When the response to risperidone is measured by GAF, and said response is a therapeutically effective response comprising an improvement of 20 points or more in GAF scales, algorithms are provided which predict a patients responsiveness. For example, in one aspect, determining the likelihood of a general response to risperidone treatment in a patient (LoR) is calculated according to the following algorithm: Likelihood of response (LoR)=[1-(−0.615−0.723A1−0.917A2+0.890B1−0.961C1+1.057C2)] where A1=α1A Arg492/Arg492, A2=α1A Arg492/Cys, B1=D2Taq I A2/A2, C1=D4−521 C/C C2=D4−521 C/T, as measured by GAF. Specifically in another aspect, the likelihood of a response to risperidone treatment (LoR) in said patient can be predicted using the following algorithm: LoR=[1−(−0.185−1.07A1−1.494A2+0.798B1−0.301C1+0.81C2+1.982D1+0.527D2−21.389E1+0.409E2−2.566F1−0.627F2)], where A1=α1A Arg492/Arg492, A2=α1A Arg492/Cys, B1=D2 A2/A2, C1=D4−521 C/C, C2=D4−521 C/T, D1=5-HT1A−1018 C/C, D2=5-HT1A−1018 C/G, E1=CYP2D6*4 A/A, E2=CYP2D6*4 A/G, F1=5-HT2A 102 T/T, and F2=5-HT2A 102 T/C, as measured by GAF.

The sequences of the polymorphic genotypes in this algorithm are listed in Table 1B below.

TABLE 1B
5-HT1A −1018 G/C GeneID: 3350
Rs6295
(SEQ ID NO: 60)
cgaGAACGGAGGTAGCTTTTTAAAAA[C/G]GAAGACACACTCGGTCTTC
TTCCAT
5-HT2A 102 T/C GeneID: 3356
Rs6313
(SEQ ID NO: 61)
AGGCTCTACAGTAATGACTTTAACTC[C/T]GGAGAAGCTAACACTTCTG
ATGCAT
Alpha 1A Arg492Cys (also described as Arg 347Cys)
GeneID: 148
Rs1048101
(SEQ ID NO: 62)
AGAATGTCTTGAGAATCCAGTGTCTC[C/T]GCAGAAAGCAGTCTTCCAA
ACATGC
CYP2D6*4 GeneID: 1565
Rs3892097
(SEQ ID NO: 63)
CCCTTACCCGCATCTCCCACCCCCA[A/G]GACGCCCCTTTCGCCCCAAC
GGTCT
D2 Taq IA GeneID: 1813
Rs1800497
(SEQ ID NO: 64)
TGGACGTCCAGCTGGGCGCCTGCCT[C/T]GACCAGCACTTTGAGGATGG
CTGTG
D4 −521 C/T GeneID: 1815
Rs1800955
(SEQ ID NO: 65)
GGCAGGGGGAGCGGGCGTGGAGGG[C/T]GCGCACGAGGTCGAGGCGAGT
CCG

### Improvement in Positive Symptoms by Risperidone Treatment

In another embodiment, there is described herein a method of determining the likelihood of improvement in positive symptoms by risperidone treatment in a patient. Positive symptoms can be assessed clinically using, for instance, the PANSS positive scale. A reduction of at least 30% in positive PANSS scores is indicative of an improvement in positive symptoms.

Predictions of responsiveness as measured by the PANSS positive scale can be made by detecting the allelic forms of one or more polymorphisms in one, two, three, or all four of the following genes of the patient: COMT, DRD2, DRD4, and 5-HT2C. In an aspect of this embodiment, the one or more polymorphisms in COMT includes Val158Met, the one or more polymorphisms in DRD2 includes Taq I A1/A2, the one or more polymorphisms in DRD4 includes −521 C/T, and the one or more polymorphisms in 5-HT2C includes −145964 A/C. In another aspect, the allelic forms of the polymorphisms detected consist of the Val158/MetMet polymorphism in COMT, the Taq I A2/A2 in D2, the −521 C/T polymorphism in D4, and the −145964 A/C polymorphism in 5-HT2C.

In one aspect, detecting the allelic forms of the polymorphisms further includes determining the copy number of the wild type allele with respect to each polymorphism. Specifically in one aspect, the likelihood of improvement in positive symptoms in response to risperidone treatment in said patient (LoR) is calculated according to the following algorithm: [1−(0.284−3.02A1−1.704A2+0.456B1+1.712C1+2.259C2−0.638D1)], wherein A1=COMT Val158/Val158, A2=COMT Val158/Met, B1=D2 Taq I A2/A2, C1=D4−521 C/C, C2=D4−521 C/T, and D1=5-HT2C−145964 A/A. The sequences of the polymorphic genotypes in this algorithm are listed in Table 1C below.

TABLE 1C
RISPERIDONE POSITIVE PANSS RESPONSE

COMT Val108/158Met GeneID: 1312
Rs4680
(SEQ ID NO: 66)
CCCAGCGGATGGTGGATTTCGCTGGC[A/G]TGAAGGACAAGGTGTGCAT
GCCTG
D2 Taq IA GeneID: 1813
Rs1800497
(SEQ ID NO: 67)
TGGACGTCCAGCTGGGCGCCTGCCT[C/T]GACCAGCACTTTGAGGATGG
CTGTG
D4 −521 C/T GeneID: 1815
Rs1800955
(SEQ ID NO: 68)
GGCAGGGGGAGCGGGCGTGGAGGG[C/T]GCGCACGAGGTCGAGGCGAGT
CCG
5-HT2C −145964 A/C GeneID: 3358
Rs475717
(SEQ ID NO: 69)
TTTTTTTTTTTTTTCTTATTTACCAC[A/C]GGACATAAATGCAAGGAAT
TTTGAT

### Improvement in Negative Symptoms by Risperidone Treatment

In another embodiment, there is described herein a method of determining the likelihood of improvement in negative symptoms by risperidone treatment in a patient. Negative symptoms can be assessed clinically using, for instance the PANSS negative scale. A reduction of at least 30% in negative PANSS scores is indicative of an improvement in negative symptoms.

Predictions of responsiveness as measured by the PANSS negative scale can be made by detecting one or more polymorphisms in one, two, three or four of the following genes of said patient: 5-HT2C, ChAT, M1 and NRG1. In an aspect of this embodiment, the one or more polymorphisms in 5-HT2C comprises −145964 A/C, the one or more polymorphisms in ChAT comprises rs1880676 G/A, the one or more polymorphisms in M1 comprises −12064 T/C, and the one or more polymorphisms in NRG1 comprises SNP8NRG221533 C/T. In another aspect, the allelic forms of the polymorphisms detected consist of the 145964 A/C polymorphism in 5-HT2C, the rs1880676 G/A polymorphism in ChAT, the −12064 T/C polymorphism in M1, and the SNP8NRG221533 C/T polymorphism in NRG1.

In one aspect, detecting the allelic forms of the polymorphisms further includes determining the copy number of the wild type allele with respect to each polymorphism. Specifically in one aspect, the likelihood of improvement in negative symptoms by risperidone treatment in said patient (LoR) is calculated according to the following algorithm: [1−(−0.076+1.451A1+3.576B1+2.944B2−0.309C1−1.17 C2−2.321D1−1.931D2)], where A1=5-HT2C rs475717−145964 A/A, B1=ChAT G/G, B2=ChAT G/A, C1=M1 12064 T/T, C2=M1−12064 T/C, D1=(NRG1) SNP8NRG221533 C/C, and D2=(NRG1) SNP8NRG221533 C/T. The sequences of the polymorphic genotypes in this algorithm are listed in Table 1D below.

TABLE 1D
RISPERIDONE NEGATIVE PANSS RESPONSE

5-HT2C −145964 A/C GeneID: 3358
Rs475717
(SEQ ID NO: 70)
TTTTTTTTTTTTTTCTTATTTACCAC[A/C]GGACATAAATGCAAGGAAT
TTTGAT
M1 −12,064 T/C GeneID: 1128
Rs12295208
(SEQ ID NO: 71)
CTGGGGGGCCGTTTGCCCTAGAGATG[C/T]GGGTCCTGCACCGCCTCTG
TTTGG
CHAT rs1880676 G/A GeneID: 1103
Rs1880676
(SEQ ID NO: 72)
CACCAGAGATGIGGCCGGAATGCAGA[A/G]ATGAAGCACTGAGCACAGT
AGGTA
Neuregulin 1 SNP8NRG221533 T/C GeneID: 3084
SNP8NRG221533
(SEQ ID NO: 73)
ACTAAAAAAGAGATATATGATATTTGG[C/T]AAAATAAAGATACATGGC
TTCCAG

### Improvement in General Psychopathology Symptom Response

In another embodiment, there is described herein a method of determining the likelihood of improvement in general psychopathology symptoms by risperidone treatment in a patient. General Psychopathology symptoms can be assessed clinically using, for instance the PANSS general psychopathology subscale. A reduction of at least 30% is indicative of an improvement in general psychopathology symptoms.

Predictions of responsiveness as measured by the PANSS general psychopathology subscale can be made by detecting one or more polymorphisms in a plurality of genes comprising one, two or three of the following genes of the patient: ChAT, 5-HT2A and NRG1. In an aspect of this embodiment, the one or more polymorphisms in ChAT comprises G/A, the one or more polymorphisms in 5-HT2A comprises rs6313 102 T/C, and the one or more polymorphisms in NRG1 comprises SNP8NRG221533 C/T. In another aspect, the allelic forms of the polymorphisms detected consist of the G/A polymorphism in ChAT, the rs6313 102 T/C polymorphism in 5HT2A and the SNP8NRG221533 C/T polymorphism of NRG1.

In one aspect, detecting the polymorphisms further includes determining the copy number of the wild type allele with respect to each polymorphism. Specifically in one aspect, the likelihood of improvement in general psychopathology in response to risperidone treatment in said patient (LoR) is calculated according to the following algorithm: =[1−(0.512+0.196A1−1.053A2−1.183B1+0.407B2+1.364C1+0.54C2)] where A1=ChAT G/G, where A2=ChAT G/A, where B1=5-HT2A 102 T/T, where B2=5-HT2AT/C, where C1=(NRG1) SNP8NRG221533 C/C, and where C2=(NRG1) SNP8NRG221533 C/T. The sequences of the polymorphic genotypes in this algorithm are listed in Table 1E below.

TABLE 1E
RISPERIDONE GP PANSS RESPONSE

Neuregulin 1 SBP8NRG221533 T/C GeneID: 3084
SNP8NRG221533
(SEQ ID NO: 74)
ACTAAAAAAGAGATATATGATATTTGG[C/T]AAAATAAAGATACATGGC
TTCCAG
5-HT2A 102 T/C GeneID: 1103
Rs6313
(SEQ ID NO: 75)
AGGCTCTACAGTAATGACTTTAACTC[C/T]GGAGAAGCTAACACTTCTG
ATGCAT
CHAT rs1880676 G/A GeneID: 1103
Rs1880676
(SEQ ID NO: 76)
CACCAGAGATGTGGCCGGAATGCAGA[A/G]ATGAAGCACTGAGCACAGT
AGGTA

In another aspect, there are provided nucleotide sequences encoding any of the above polymorphisms as described herein. Specifically, nucleic acids comprising the allelic forms encoding Arg492Cys and −6274 T/C of ADRA1A, the allelic forms of Taq I A2/A1 of DRD2, the allelic forms of 2630 T/C in 5-HTT, the allelic forms of *4 EM/PM of CYP2D6, the allelic forms of −1018-C/G in 5-HT1A, the allelic forms of −1438-G/A in 5-HT2A, the allelic forms at the loci encoding Cys23Ser in 5-HT2C, the allelic forms of −521 C/T in DDR4, the allelic forms of rs6313 102 T/C in 5-HT2A, the Val158Met polymorphism in COMT, the −145964 A/C polymorphism in 5-HT2C, the rs1880676 G/A polymorphism in ChAT, the −12064 T/C polymorphism in M1, and the SNP8NRG221533 C/T polymorphism in NRG1.

Also described herein is a kit for determining a genotype of an individual, which comprises one or more oligonucleotides that enable detection of a combination of polymorphisms described herein. In one embodiment the genotype of the polymorphisms listed in Tables 1A-1E can be detected using the oligonucleotides listed in Table 2.

TABLE 2
Algorithm SNP Primer Sequences
All sequences 5′-3′
ADRA1A Arg492Cys (also described as Arg347Cys)

| | | |
|---|---|---|
| F: | ATG CTC CAG CCA AGA GTT CA | (SEQ ID NO: 3) |
| R: | TCC AAG AAG AGC TGG CCT TC | (SEQ ID NO: 4) |

ADRA1A −6274

| | | |
|---|---|---|
| F: | TAT GTA TAT GCT ATT GTC TGA AAG | (SEQ ID NO: 5) |
| R: | AAG CGC CCA TTC TTC ATA GA | (SEQ ID NO: 6) |

CHAT rs1880676

| | | |
|---|---|---|
| Allele 1: | AGA TGT GGC CGG AAT GCA GAG | (SEQ ID NO: 11) |
| Allele 2: | GAG ATG TGG CCG GAA TGC AGA A | (SEQ ID NO: 12) |
| Common: | CATACCTACTGTGCTCAGTGCTTCAT | (SEQ ID NO: 13) |

COMT Val102/158Met

| | | |
|---|---|---|
| F: | TCG TGG ACG CCG TGA TTC AGG | (SEQ ID NO: 16) |
| R: | AGG TCT GAC AAC GGG TCA GGC | (SEQ ID NO: 17) |

CYP2D6*4

| | | |
|---|---|---|
| F: | GCC TTC GCC AAC CAC TCC G | (SEQ ID NO: 18) |
| R: | AAA TCC TGC TCT TCC GAG GC | (SEQ ID NO: 19) |

D2 Taq IA

| | | |
|---|---|---|
| F: | CCG TCG ACG GCT GGC CAA GTT GTC TA | (SEQ ID NO: 20) |
| R: | CCG TCG ACC CTT CCT GAG TGT CAT CA | (SEQ ID NO: 21) |

D4 −521 C/T

| | | |
|---|---|---|
| F: | GCA TCG ACG CCA GCG CCA TCC TAC C | (SEQ ID NO: 24) |
| R: | ATG AGC TAG GCG TCG GCG G | (SEQ ID NO: 25) |

5-HT1A −1018 G/C

| | | |
|---|---|---|
| F: | TGG AAG AAG ACC GAG TGT GTC TAC | (SEQ ID NO: 26) |
| R: | TTC TCC CTG GGA GAG TAA GGC TGG | (SEQ ID NO: 27) |

5-HT2A 102 T/C

| | | |
|---|---|---|
| F: | TCT GCT ACA AGT TCT GGC TT | (SEQ ID NO: 28) |
| R: | CTG CAG CTT TTT CTC TAG GG | (SEQ ID NO: 29) |

5-HT2A −1438 G/A

| | | |
|---|---|---|
| F: | AAG CTG CAA GGT AGC AAC AGC | (SEQ ID NO: 30) |
| R: | AAC CAA CTT ATT TCC TAC CAC | (SEQ ID NO: 31) |

5-HT2C rs475717 A/C

| | | |
|---|---|---|
| F: | CGA AGG CAG GTA TTT TCA CA | (SEQ ID NO: 32) |
| R: | TGG ATC TGA TGC TGG GTT TT | (SEQ ID NO: 33) |

5-HT2C Cys23Ser

| | | |
|---|---|---|
| F: | TTG GCC TAT TGG TTT GGG AAT | (SEQ ID NO: 34) |
| R: | GTC TGG GAA TTT GAA GCG TCC AC | (SEQ ID NO: 35) |

5-HTT (SLC6A4) 2630 C/T

| | | |
|---|---|---|
| F: | TTT CAG AGG AGG CCA AGA GA | (SEQ ID NO: 38) |
| R: | TTC AGG AGG TCT GGG GTA GA | (SEQ ID NO: 39) |

M1 −12,064 T/C

| | | |
|---|---|---|
| F: | GTGAGAAAGCCCCAGGTTAC | (SEQ ID NO: 42) |
| R: | CCAGGCTGGTCTGGACTTCTG | (SEQ ID NO: 43) |

Neuregulin 1 221533

| | | |
|---|---|---|
| F: | AAG GCA TCA GTT TTC AAT AGC TTT TT | (SEQ ID NO: 44) |
| R: | TAA GTA GAA ATG GGA ACT CTC CAT CTC | (SEQ ID NO: 45) |
| Probe 1: | FAM-TTT ATT TTg CCA AAT AT-MGB | (SEQ ID NO: 46) |
| Probe 2: | VIC-TCT TTA TTT TaC CAA ATA TCA T-MGB | (SEQ ID NO: 47) |

In an embodiment of the methods described herein, the procedure for detecting the allelic forms of the polymorphisms is preferably, but not limited to, a procedure selected from the group of: DNA sequencing, allele-specific amplification, and allele-specific primer extension. However, any procedure for detecting the allelic forms of the polymorphisms is encompassed by the invention, including, but preferably not limited to, single strand conformation polymorphism (SSCP), denaturing gradient gel electrophoresis (DGGE) or temperature gradient gel electrophoresis analysis (TGGE), mismatch cleavage analysis, cleavage-fragment-length polymorphism analysis (CFLP), denaturing high pressure liquid chromatography (dHPLC), chemical cleavage of mismatch (CCM), Enzymatic cleavage of mismatch (ECM), UNG-mediated T Scan, direct sequencing, DNA chip resequencing, and Pyrosequencing™.

In one embodiment, there is a kit for determining a genotype of an individual, which comprises one or more oligonucleotides that enable detection of one or more or all of the following genotypes at the following polymorphisms selected from the group consisting of: 5-HTT 2630-T/T genotype, 5-HTT 2630-T/C genotype, D2 Taq I A2/A2 genotype, D2 Taq I A2/A1 genotype, α1A Arg492/Arg492 genotype and α1A Arg492/Cys genotype. In one aspect, the oligonucleotides of the kit comprise oligonucleotides with the following sequences: SEQ ID NO:38, SEQ ID NO:39, SEQ ID NO:20, SEQ ID NO:21, SEQ ID NO:3, and SEQ ID NO:4.

In another embodiment, there is a kit for determining a genotype of an individual, which comprises one or more oligonucleotides that enable detection of one or more or all of the following genotypes at the following polymorphisms selected from the group consisting of: 5-HTT 2630-T/C genotype, D2 TaqI A2/A2 genotype, D2 TaqI A2/A1 genotype, α1A Arg492/Arg492 genotype, α1A Arg492/Cys genotype, α1A−6274-C/C genotype, CYP2D6*4 EM/EM genotype, CYPD6*4 EM/PM genotype, 5-HT1A−1018-C/C genotype, 5-HT1A−1018-C/G genotype, 5-HT2A−1438-G/G genotype, 5-HT2A−1438-G/A genotype, and 5-HT2C Cys23Ser/Cys23Ser or Cys23Ser genotypes. In one aspect, the oligonucleotides of the kit comprise oligonucleotides with the following sequences: SEQ ID NO:38, SEQ ID NO:39, SEQ ID NO:20, SEQ ID NO:21, SEQ ID NO:3, SEQ ID NO:4, SEQ ID NO:5, SEQ ID NO:6, SEQ ID NO:18, SEQ ID NO:19, SEQ ID NO:26, SEQ ID NO:27, SEQ ID NO:30, SEQ ID NO:31, SEQ ID NO:34 and SEQ ID NO:35.

In another embodiment, there is a kit for determining a genotype of an individual, which comprises one or more oligonucleotides that enable detection of one or more or all of the following genotypes at the following polymorphisms selected from the group consisting of: α1A Arg492/Arg492, α1A Arg492/Cys, D2 Taq I A2/A2, D4−521 C/C and D4−521 C/T. In one aspect, the oligonucleotides of the kit comprise oligonucleotides with the following sequences: SEQ ID NO:3, SEQ ID NO:4, SEQ ID NO:20, SEQ ID NO:21, SEQ ID NO:24 and SEQ ID NO:25.

In another embodiment, there is a kit for determining a genotype of an individual, which comprises one or more oligonucleotides that enable detection of one or more or all of the following genotypes at the following polymorphisms selected from the group consisting of: α1A Arg492/Arg492, α1A Arg492/Cys, D2 Taq I A2/A2, D4−521 C/C and D4−521 C/T, 5-HT1A−1018 C/C, 5-HT1A−1018 C/G, CYP2D6*4 A/A, CYP2D6*4 A/G, 5-HT2A 102 T/T, and 5-HT2A 102 T/C. In one aspect, the oligonucleotides of the kit comprise oligonucleotides with the following sequences: SEQ ID NO:3, SEQ ID NO:4, SEQ ID NO:20, SEQ ID NO:21, SEQ ID NO:24, SEQ ID NO:25, SEQ ID NO:26, SEQ ID NO:27, SEQ ID NO:18, SEQ ID NO:19, SEQ ID NO:28 and SEQ ID NO:29.

Metgenotypesgenotypes In another embodiment, there is a kit for determining a genotype of an individual, which comprises one or more oligonucleotides that enable detection of one or more or all of the following genotypes at the following polymorphisms selected from the group consisting of: COMT rs4680 Val158/Val158, COMT rs4680 Val158/Met, D2 Taq I A2/A2, D4−521 C/C, D4−521 C/T, and 5-HT2C−145964 A/A. In one aspect, the oligonucleotides of the kit comprise oligonucleotides with the following sequences: SEQ ID NO:16, SEQ ID NO:17, SEQ ID NO:20, SEQ ID NO:21, SEQ ID NO:24, SEQ ID NO:25, SEQ ID NO:32 and SEQ ID NO:33.

In another embodiment, there is a kit for determining a genotype of an individual, which comprises one or more oligonucleotides that enable detection of one or more or all of the following genotypes at the following polymorphisms selected from the group consisting of: 5-HT2C−145964 A/A, ChAT rs1880676 G/G, ChAT rs1880676 G/A, M1−12064 T/T, M1−12064 T/C, NRG1 SNP8NRG221533 C/C, and NRG1 SNP8NRG221533 C/T. In one aspect, the oligonucleotides of the kit comprise oligonucleotides with the following sequences: SEQ ID NO:32, SEQ ID NO:33, SEQ ID NO:11, SEQ ID NO:12, SEQ ID NO:13, SEQ ID NO:42, SEQ ID NO:43, SEQ ID NO:44, SEQ ID NO:45, SEQ ID NO:46 and SEQ ID NO:47.

In another embodiment, there is a kit for determining a genotype of an individual, which comprises one or more oligonucleotides that enable detection of one or more or all of the following genotypes at the following polymorphisms selected from the group consisting of: ChAT rs1880676 G/G, ChAT rs1880676 G/A, 5-HT2A 102 T/T, 5-HT2A 102 T/C, NRG1 SNP8NRG221533 C/C, and NRG1 SNP8NRG221533 C/T. In one aspect, the oligonucleotides of the kit comprise oligonucleotides with the following sequences: SEQ ID NO:11, SEQ ID NO:12, SEQ ID NO:13, SEQ ID NO:28, SEQ ID NO:29, SEQ ID NO:44, SEQ ID NO:45, SEQ ID NO:46, SEQ ID NO:47.

### DETAILED DESCRIPTION

### Definitions

As will become apparent, preferred features and characteristics of one aspect of the invention are applicable to any other aspect of the invention. It should be noted that, as used herein, the singular form “a”, “an” and “the” include plural references unless the context clearly dictates otherwise.

Psychosis: Patients suffering from psychosis have impaired reality testing; that is, they are unable to distinguish personal, subjective experience from the reality of the external world. They experience hallucinations and/or delusions that they believe are real, and may behave and communicate in an inappropriate and incoherent fashion. Psychosis may appear as a symptom of a number of mental disorders and it is the defining feature of schizophrenia, schizophreniform disorder, schizoaffective disorder, delusional disorder, and the psychotic disorders (i.e., brief psychotic disorder, shared psychotic disorder, psychotic disorder due to a general medical condition, and substance-induced psychotic disorder). Functional causes of psychosis also include “mood disorders such as bipolar disorder (manic depression) and severe clinical depression.

As used herein, the term “schizophrenia” is intended to include the group of mental disorders characterized by disruptions in thinking and perception. In a clinical evaluation, schizophrenia is commonly marked by “positive symptoms” such as auditory hallucinations (especially hearing voices), disorganized thought processes and delusions as well as “negative symptoms” which include affective flattening, alogia, avolition, and anhedonia.

As used herein “the positive symptoms of schizophrenia” refer to a class of symptoms of schizophrenia characterised by hallucinations (sensory perception in the absence of external stimuli) which may occur in any of the five senses, although tend to be auditory. They are a common feature of psychosis and are prominent and often distressing. Positive symptoms also include delusional or paranoid false beliefs that are usually incomprehensible in terms of normal mental processes yet held with conviction by the patient. Thought disorder describes an underlying disturbance to conscious thought and is classified largely by its effects on speech and writing.

As used herein, “the negative symptoms of schizophrenia” refer to a class of symptoms of schizophrenia which can be considered to reflect a ‘loss’ in functional, directed thought or activity. Negative symptoms of schizophrenia are well known in the art, and include affective flattening (characterized by, for example, an immobile and/or unresponsive facial expression, poor eye contact and reduced body language), alogia (‘poverty of speech’ or brief, laconic and/or empty replies), avolition (characterized by a reduced or absent ability to initiate and carry out goal-directed activities), anhedonia (loss of interest or pleasure), social withdrawal, apathy and other negative symptoms known to those of skill in the art.

The symptoms and general functioning of patients with schizophrenia are assessed using a variety of published rating scales. Ratings may be made in terms of overall symptoms or in terms of level of functioning, or in terms of specific symptom groups.

The negative symptoms of schizophrenia may be assessed using any methodology known in the art including, but not limited to, the Brief Psychiatric Rating Scale (BPRS), the Positive and Negative Syndrome Scale (PANSS), the Rorschach Schizophrenia Index (SCZI), and the Scale for the Assessment of Negative Symptoms (SANS). Some of these methods may also be used to assess positive symptoms (e.g., BPRS and PANSS), although methods for specifically assessing positive symptoms are also available (e.g., the Scale for the Assessment of Positive Symptoms, or SAPS).

General symptoms of psychopathology associated with psychotic illness (such as somatic concern anxiety, guilt feelings, tension) may also be assessed (e.g. by the PANSS scale). The symptoms or symptom classes/subgroups of psychosis may be assessed separately e.g., as individual scale items, or as subscales, (e.g., negative symptom scale of PANSS, positive symptom scale of PANSS, general psychopathology scale of PANSS,) or combined to give a total overall assessment of symptoms (e.g. Total PANSS score).

An overall assessment of symptoms and functioning may be obtained by other scales including but not limited to the GAF (Global assessment of Functioning scale) and the CGI (Clinical Global Impression scale).

As used herein, the terms “response to risperidone treatment” includes pharmacological effectiveness. Pharmacological effectiveness refers to the ability of the treatment to result in a desired biological effect in the patient.

As used herein “risperidone treatment” refers to a course of treatment encompassing administration of risperidone to a patient in therapeutically effective amount(s) over a time period. In one embodiment the time period is three months or more, up to and including, 6 months, a year, three years or longer. However the time period can also be shorter than three months. All the methods of treating described herein include administration of risperidone or a risperidone related molecule—by any method known to those skilled in the art including subcutaneous, intramuscular, intradermal, transdermal, intraperitoneal, intravenous, intranasal, intrathecal, intraocular, or oral routes of administration.

In one embodiment, a psychotic patient's response to risperidone treatment response was assessed prospectively using the PANSS and GAF scales. In an aspect of this embodiment, a “positive response to risperidone treatment” means a reduction in the symptoms of the psychotic disease, and in one embodiment is evidenced by an improvement of at least 20 points or more in the GAF scales, or at least 30% decrease in PANSS values after risperidone treatment. A positive response may also encompasses an improvement in specific symptoms of a psychotic disease. This may include—an improvement in positive symptoms, and/or negative symptoms and/or a general psychopathology symptoms response. The more positive the response, the more the symptoms are reduced. These patients are classified as “Responders”.

As used herein the term “a negative response to risperidone treatment” means the treatment provides no reduction of the assessed symptoms of the psychotic disease, or causes an increase in the symptoms of the psychotic disease being treated. The more negative the response, the more the symptoms are increased. These patients are classified as “nonresponders”.

A patient may be an overall responder as measured by GAF or PANSS total score, but may still, for example, be a negative symptom non-responder if the improvement in the negative symptoms to risperidone treatment fails to meet the response criteria. As used herein, the phrase “likelihood of a response” to risperidone treatment means the probability that a patient will display the response after risperidone treatment. Probability can be measured in terms of percentage, ranging from 0 to 100%: if the percentage is low, then there is a low likelihood that the patient will have the response of interest, and conversely, where the percentage is high, there is a higher likelihood or probability that the patient will display the response of interest. Expression of psychotic diseases is multifactorial, hence it is unlikely to achieve a probability of 100% based on hereditary factors alone. Accordingly, the phrase “determining the likelihood of a response” to risperidone provides an approximate probability that a patient with a particular genotype at specific polymorphic loci will display the response to risperidone being measured.

As used herein, the phrase: “Improvement in negative symptoms” corresponds to at least a 30% decrease in negative PANSS scores

As used herein, the phrase: “Improvement in positive symptoms” corresponds to at least a 30% decrease in positive PANSS scores

As used herein, the phrase: “Improvement in general psychopathology symptoms” corresponds to at least 30% decrease in general psychopathology PANSS scores. The term “genotype” in the context of this invention refers to the particular allelic forms of a gene, which can be defined by the particular nucleotide(s) present in a nucleic acid sequence at a particular site(s).

The terms “polymorphism”, “genotype”, “variant form of a gene”, “form of a gene” or “allele” refer to one specific form of a gene in a population, the specific form differing from other forms of the same gene in the sequence of at least one, and frequently more than one, variations from wild type within the sequence of the gene. The sequences at these sites of variation within a gene that differ between alleles of the gene are termed “gene sequence polymorphisms” or “polymorphisms” or “variants” or “allelic variants”. Other terms known in the art to be equivalent include mutation and polymorphism. The polymorphisms may be single or multiple base changes, including without limitation insertions, deletions, or substitutions, or may be a variable number of sequence repeats.

In one aspect, the term “Allele” refers to normal alleles of a locus as well as alleles of the gene carrying variations that affect responsiveness to risperidone. In preferred aspects of this invention, the polymorphisms are selected from the group consisting of the polymorphisms listed in Table 1.

“Isolated” or “substantially pure”. An “isolated” or “substantially pure” nucleic acid (e.g., an RNA, DNA or a mixed polymer) is one which is substantially separated from other cellular components which naturally accompany a native human sequence or protein, e.g., ribosomes, polymerases, many other human genome sequences and proteins. The term embraces a nucleic acid sequence or protein which has been removed from its naturally occurring environment, and includes recombinant or cloned DNA isolates and chemically synthesized analogs or analogs biologically synthesized by heterologous systems.

“Encode” A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced there from.

The term “promoter sequence” or “promoter” defines a single strand of a nucleic acid sequence that is specifically recognized by an RNA polymerase that binds to a recognized sequence and initiates the process of transcription by which an RNA transcript is produced. In principle, any promoter sequence may be employed for which there is a known and available polymerase that is capable of recognizing the initiation sequence. Known and useful promoters are those that are recognized by certain bacteriophage polymerases, such as bacteriophage T3, T7 or SP6.

“Regulatory sequences” refers to those sequences normally within 100 kb of the coding region of a gene, but they may also be more distant from the coding region, which affect the expression of the gene (including transcription of the gene, and translation, splicing, stability or the like of the messenger RNA).

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

A “microarray” is a linear or two-dimensional array of preferably discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm² even more preferably at least about 500/cm², and still more preferably at least about 1,000/cm². As used herein, a DNA microarray is an array of oligonucleotide primers placed on a chip or other surfaces used to amplify or clone target polynucleotides. Since the position of each particular group of primers in the array is known, the identities of the target polynucleotides can be determined based on their binding to a particular position in the microarray.

As used herein, a “sample” refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), organs, and also samples of in vitro cell culture constituent.

The term “amplify” is used in the broad sense to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases. Any of several techniques that increase the number of copies of a nucleic acid molecule. A preferred example of amplification is the polymerase chain reaction (PCR), in which a sample containing the nucleic acid is contacted with a pair of oligonucleotide primers under conditions that allow for the hybridization of the primers to nucleic acid in the sample. The primers are extended under suitable conditions, dissociated from the template, and then re-annealed, extended, and dissociated to amplify the number of copies of the nucleic acid. The amplification products (called “amplicons”) can be further processed, manipulated, or characterized by (without limitation) electrophoresis, restriction endonuclease digestion, hybridization, nucleic acid sequencing, ligation, or other techniques of molecular biology. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; liGAFe chain reaction amplification, as disclosed in European Patent Appl. 320 308; gap filling liGAFe chain reaction amplification, as disclosed in U.S. Pat. No. 5,427,930; and RNA transcription-free amplification, as disclosed in U.S. Pat. No. 6,025,134.

The term “primer”, as used herein, refers to an oligonucleotide which is capable of acting as a point of initiation of polynucleotide synthesis along a complementary strand when placed under conditions in which synthesis of a primer extension product which is complementary to a polynucleotide is catalyzed. Such conditions include the presence of four different nucleotide triphosphates or nucleoside analogs and one or more agents for polymerization such as DNA polymerase and/or reverse transcriptase, in an appropriate buffer (“buffer” includes substituents which are cofactors, or which affect pH, ionic strength, etc.), and at a suitable temperature. A primer must be sufficiently long to prime the synthesis of extension products in the presence of an agent for polymerase. A typical primer contains at least 5 nucleotides in length of a sequence substantially complementary to the target sequence, but somewhat longer primers are preferred. Usually primers contain about 15−26 nucleotides, but longer primers may also be used.

A primer will always contain a sequence substantially complementary to the target sequence, that is the specific sequence to be amplified, to which it can anneal. A primer may, optionally, also comprise a promoter sequence. Primers are useful to amplify sequences from the region of the polymorphism and are preferably complementary to, and hybridize specifically to, sequences that flank one or more polymorphisms in a gene. Polymorphic sequences generated by amplification may be sequenced directly or may be cloned prior to sequence analysis. A method for the direct cloning and sequence analysis of enzymatically amplified genomic segments has been described by Scharf et al., 1986.

In the context of this invention, the term “probe” refers to a molecule which can detectably distinguish between target molecules differing in structure, such as allelic variants. Detection can be accomplished in a variety of different ways but preferably is based on detection of specific binding. Examples of such specific binding include antibody binding and nucleic acid probe hybridization. Thus, for example, probes can include enzyme substrates, antibodies and antibody fragments, and preferably nucleic acid hybridization probes.

“Polynucleotide Probes”. Polynucleotide polymorphisms associated with genotypes which contribute to the sensitivity of a patient's response to risperidone treatment can be detected by hybridization with a polynucleotide probe which forms a stable hybrid with that of the target sequence, under stringent to moderately stringent hybridization and wash conditions. If it is expected that the probes will be perfectly complementary to the target sequence, high stringency conditions will be used. Hybridization stringency may be lessened if some mismatching is expected, for example, if variants are expected with the result that the probe will not be completely complementary. Conditions are chosen which rule out nonspecific/adventitious bindings, that is, which minimize noise. For techniques for preparing and labeling probes see, e.g., Sambrook et al., 1989 or Ausubel et al., 1992. Probes comprising synthetic oligonucleotides or other polynucleotides of the present invention may be derived from naturally occurring or recombinant single- or double-stranded polynucleotides, or be chemically synthesized. Probes may also be labeled by nick translation, Klenow fill-in reaction, or other methods known in the art.

The term “gene” as used herein is a polynucleotide which may include coding sequences, intervening sequences and regulatory elements controlling transcription and/or translation. The term “gene” as used herein is intended to encompass all allelic variations of the gene's DNA sequence. Genes of the invention refer to those genes that are likely to be expressed in normal tissue, certain alleles of which contribute to a patient's response to risperidone. As used herein a gene encompasses a polynucleotide which encodes a polypeptide, fragment, homolog or variant, including, e.g., protein fusions or deletions or insertions. The nucleic acids of the present invention will possess a sequence which is either derived from, or has substantial homology with a natural encoding gene which contributes to a patient's response to risperidone, or a portion thereof.

Genes of the invention include normal alleles of the gene encoding polymorphisms that contribute to a patient's sensitivity to risperidone, including silent alleles having no effect on the amino acid sequence of the gene's encoded polypeptide as well as alleles leading to amino acid sequence variants of the encoded polypeptide that do not substantially affect its function or its contribution to responsiveness to risperidone therapy. These terms also include alleles having one or more mutations which affect the function of the encoded polypeptides and it's contribution to responsiveness to risperidone therapy.

The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The present invention provides recombinant nucleic acids comprising all or part of a gene encoding a polymorphism contributing to the sensitivity of a patient's response to risperidone treatment. The recombinant construct may be capable of replicating autonomously in a host cell. Alternatively, the recombinant construct may become integrated into the chromosomal DNA of the host cell. Such a recombinant polynucleotide comprises a polynucleotide of genomic, cDNA, semi-synthetic, or synthetic origin which, by virtue of its origin or manipulation, 1) is not associated with all or a portion of a polynucleotide with which it is associated in nature; 2) is linked to a polynucleotide other than that to which it is linked in nature; or 3) does not occur in nature. Where nucleic acid according to the invention includes RNA, reference to the sequence shown should be construed as reference to the RNA equivalent, with U substituted for T.

Therefore, recombinant nucleic acids comprising sequences otherwise not naturally occurring are provided by this invention. Although the wild-type sequence may be employed, it will often be altered, e.g., by deletion, substitution or insertion. cDNA or genomic libraries of various types may be screened as natural sources of the nucleic acids of the present invention, or such nucleic acids may be provided by amplification of sequences resident in genomic DNA or other natural sources, e.g., by PCR. The choice of cDNA libraries normally corresponds to a tissue source which is abundant in mRNA for the desired proteins. Phage libraries are normally preferred, but other types of libraries may be used. Clones of a library are spread onto plates, transferred to a substrate for screening, denatured and probed for the presence of desired sequences. The methods of nucleic acid isolation, amplification and analysis are routine for one skilled in the art and examples of protocols can be found, for example, in the Molecular Cloning: A Laboratory Manual (3-Volume Set) Ed. Joseph Sambrook, David W. Russel, and Joe Sambrook, Cold Spring Harbor Laboratory; 3rd edition (Jan. 15, 2001), ISBN: 0879695773. Particularly useful protocol source for methods used in PCR amplification is PCR (Basics: From Background to Bench) by M. J. McPherson, S. G. Moller, R. Beynon, C. Howe, Springer Verlag; 1st edition (Oct. 15, 2000), ISBN: 0387916008.

“Substantial homology or similarity”. A nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95−98% of the nucleotide bases. To determine homology between two different nucleic acids, the percent homology is to be determined using the BLASTN program “BLAST 2 sequences”. This program is available for public use from the National Center for Biotechnology Information (NCBI) over the Internet (http://www.ncbi.nlm.nih.gov/gorf/b12.html) (Altschul et al., 1997).

“Kit” refers to a combination of physical elements, e.g., probes, including without limitation specific primers, labeled nucleic acid probes, antibodies, protein-capture agent(s), reagent(s), instruction sheet(s) and other elements useful to practice the invention, in particular identify the alleles present in a polymorphism. These physical elements can be arranged in any way suitable for carrying out the invention. For example, probes and/or primers can be provided in one or more containers or in an array or microarray device.

Methods of Detecting Alleles of Polymorphisms Associated with Risperidone

An allele associated with a response to risperidone treatment can be detected by any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Preferred amplification methods are selected from the group consisting of: the polymerase chain reaction (PCR), the liGAFe chain reaction (LCR), strand displacement amplification (SDA), cloning, and variations of the above (e.g. RT-PCR and allele specific amplification). Oligonucleotides necessary for amplification may be selected for example, from within the gene loci containing the polymorphism of interest, either flanking the polymorphism of interest (as required for PCR amplification) or directly overlapping the polymorphism of interest (as in allele specific oligonucleotide (ASO) hybridization). In a particularly preferred embodiment, the sample is hybridized with a set of primers, which hybridize 5′ and 3′ in a sense or antisense sequence to the allele of interest, and is subjected to a PCR amplification.

Allele-specific oligonucleotide (ASO) hybridization as described by Henri WAJCMAN, MD, Ph. D on the URL://rbc.gs-im3.fr/DATA/The%20HW_CD/EnglASO.html Dec. 12, 2007

Two methods for diagnosis are based on this approach:

1. The dot-blotting method requires the binding of the PCR amplified target DNA sequence to a nylon membrane. The DNA fixed to the membrane is then hybridized to the allele specific oligonucleotide probes that are 5′ end-labelled with either 32P-labelled deoxynucleoside triphosphates, biotin, horseradish peroxidase or a fluorescent marker.

For mutation screening, a panel of ASO probes is required that needs to be adapted to the mutations found in the ethnic group of the individual, which is tested.

For genotyping homozygous patients, two oligonucleotide probes are required for each mutation: one complimentary to the mutant DNA sequence and the other complimentary to the normal gene sequence at the same position.

The patient's genotype is determined by analysis of the presence or absence of the hybridisation signal of both the mutation specific and normal probe. The technique is used with great success for investigation of populations with just one common mutation and a small number of rare ones. However, this method is not adapted to screening populations carrying a large number of different mutations since each mutation requires a separate hybridization and washing step.

The reverse dot-blotting technique allows several mutations to be tested in a single hybridization reaction. In this method, in contrast to the previous one, unlabelled ASO probes, specific to various mutations and to the normal DNA sequence, are bound to a nylon membrane strips in the form of dots or slots. A labelled amplified genomic DNA is then hybridized to the filter. This procedure may require the use of several filters, the first one corresponds to the more frequent mutations observed in the patient's ethnic and the others to less frequent abnormalities.

An allele of interest may also be detected indirectly, e.g. by analyzing the protein product encoded by the DNA. For example, where the polymorphism in question results in the translation of a variable protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications. Such immunodetection methods include enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference.

Genotypes Associated with Responsiveness to Risperidone

An allele whose presence is identified with individuals responding to psychiatric treatment with risperidone, either alone or in combination with other genotypes at different polymorphisms, is encompassed herein. Examples of these types of genotypes are listed in Table 1.

### Kits and Diagnostic Products and Methods

The present invention is useful in a diagnostic product to detect the presence of risperidone sensitive allele(s). Accordingly, the invention encompasses the use of diagnostic kits based on a variety of methodologies, e.g., sequence, chip, mass-spectroscopy, which are capable of finding allelic sequences indicative of the polymorphic genotypes described herein, e.g. Table 1. The invention also provides an article of manufacturing comprising packaging material and a pharmaceutical agent contained within the packaging material, wherein the pharmaceutical agent comprises means for detecting the presence of one or more genotypes of a polymorphism associated with a risperidone response, and packaging material comprises a label or package insert which indicates that the detection means can be used to identify a candidate subject suitable for treatment of a psychiatric disease such as schizophrenia with risperidone.

The present invention therefore also provides predictive and prognostic kits comprising degenerate primers to amplify polymorphic genotypes associated with a response to risperidone in a patient and instructions comprising an amplification protocol and analysis of the results. The kit may alternatively also comprise buffers, enzymes, and containers for performing the amplification and analysis of the amplification products. The kit may also be a component of a screening or prognostic kit comprising other tools such as DNA microarrays. Preferably, the kit also provides one or more control templates, such as nucleic acids isolated from normal tissue sample, and/or a series of samples representing different polymorphisms in the same gene or in different genes associated with a response to risperidone.

The kit may also include instructions for use of the kit to amplify specific targets on a solid support. Where the kit contains a prepared solid support having a set of primers already fixed on the solid support, e.g. for amplifying a particular set of target polynucleotides, the kit also includes reagents necessary for conducting a PCR on a solid support, for example using an in situ-type or solid phase type PCR procedure where the support is capable of PCR amplification using an in situ-type PCR machine. The PCR reagents, included in the kit, include the usual PCR buffers, a thermostable polymerase (e.g. Taq DNA polymerase), nucleotides (e.g. dNTPs), and other components and labeling molecules (e.g. for direct or indirect labeling). The kits can be assembled to support practice of the PCR amplification method using immobilized primers alone or, alternatively, together with solution phase primers.

In one embodiment, the kit provides two or more primer pairs, each pair capable of amplifying a different region of a gene associated with risperidone response and/or multiple polymorphisms from a plurality of genes, thereby providing a kit for analysis of expression of several gene polymorphisms in a biological sample in one reaction or several parallel reactions. Primers in the kits may be labeled, for example fluorescently labeled, to facilitate detection of the amplification products and consequent analysis of the nucleic acid variances.

In one embodiment, more than one polymorphism can be detected in one analysis. A combination kit will therefore comprise of primers capable of amplifying different segments of a single gene. The primers may be differentially labeled, for example using different fluorescent labels, so as to differentiate between the variances.

The primers contained within the kit may include those listed in Table 2, and various subcombinations thereof.

### Method of Treating a Patient

In one embodiment, the invention provides a method for selecting a treatment for a patient affected by a psychotic disease by determining the genotype of at least one polymorphism in the patient. In a preferred embodiment, the genotype of a plurality of polymorphisms in the patient is determined, whereby a plurality may include variances from one, two, three or more gene loci. For even greater specificity, an analysis of a second subset of polymorphisms associated with a response to risperidone is undertaken.

In certain embodiments, the presence of at least one allelic variation from wild type in a polymorphism associated with risperidone treatment is indicative that the treatment will be effective or otherwise beneficial (or more likely to be beneficial) in the patient. Stating that the treatment will be effective means that the probability of beneficial therapeutic effect is greater than in a person not having the above referenced genotype variation.

Table 1A-E is a partial list of DNA sequence polymorphisms in genes relevant to the methods described in the present invention. These polymorphisms were identified by the inventors in studies of biological samples from patients with psychotic disorders who benefited from risperidone therapy.

This will require continued mutational analyses and identification of additional genes and polymorphisms which contribute to a patient's response to risperidone. With more detailed phenotypic analyses, phenotypic differences between the varied forms of patient responsiveness to risperidone, such as improvement in negative symptoms, or improvement in positive symptoms may be discovered. These differences may be useful to further modify therapeutic treatment, and extend the analysis and treatment to other populations. A “population” refers to a defined group of individuals or a group of individuals with a particular disease or condition or individuals that may be treated with a specific drug identified by, but not limited to geographic, ethnic, race, gender, and/or cultural indices. In most cases a population will preferably encompass at least ten thousand, one hundred thousand, one million, ten million, or more individuals, with the larger numbers being more preferable. In preferred embodiments of this invention, the population refers to individuals with a specific disease or condition that may be treated with a specific drug.

The present invention is further detailed in the following Examples, which are offered by way of illustration and are not intended to limit the invention in any manner. Standard techniques well known in the art or the techniques specifically described below are utilized.

### EXAMPLES

### Materials and Methods

The mathematical algorithms were calculated using information of individual genetic association studies performed on clinical samples. After gathering information on which genetic polymorphisms may be associated with treatment variability, a mathematical algorithm was produced by logistic regression that allocates a predictive value or coefficient to each relevant genetic polymorphism according to their contribution to response variability. The prediction score was obtained by multiplying the coefficients by the genotypes present in an individual. The resulting value gives an indication of the likelihood of response.

### Example 1

### Clinical samples

Risperidone clinical sample: 124 subjects (99 with schizophrenia or schizo-affective disorder, 12 with bipolar disorder, 3 with major depression and 10 with atypical psychosis) were recruited in Navarra (Northern Spain) and were of Basque and Spanish origin. All subjects were treated with the antipsychotic risperidone for a minimum of 3 months. Treatment response was assessed prospectively using the PANSS(2) and GAF scales. Improvement of 20 points or more in the GAF scales, or at least a 30% decrease in PANSS values on risperidone treatment was considered as the threshold for response.

DNA was extracted from whole blood samples using standard methods.

### Example 2

### Genotyping of Predictive Polymorphisms

Polymorphisms of interest were genotyped using PCR amplification using the primers and conditions described in Table 2. The skilled person understands that the conditions and protocols used for the detection of the predictive polymorphisms are not relevant to the invention and can be easily modified and adapted to accommodate the systems/technology available in most laboratories.

### Example 3

### Calculation of Prediction Algorithms

The clinical samples were genotyped for all the polymorphisms of interest listed in Table 1. This information was then combined to produce a predictive algorithm for risperidone as follows: Logistic regression was calculated considering response to risperidone treatment as the predicted bimodal (response or non-response) variable. For algorithms predicting improvement in overall positive, negative or general psychopathology symptoms, bimodal variables based on an at least 30% decrease in overall positive, negative or general psychopathology symptoms were created respectively. Logistic regression was then calculated using the appropriate response variable. After performing logistic regression analyses, an algorithm was produced in which the genotypes of the predictor polymorphisms were multiplied by a coefficient according to their contribution to response variability. The equation algorithm is described below:

### Example 4

### Equation Algorithm for Genetic Prediction of Response to Risperidone, as Assessed on the GAF Scale

Logistic regression on risperidone response, calculated on the risperidone sample described above, and using as predictor variables genetic polymorphisms in the alpha-1A adrenergic receptor (α_{1A}), the dopamine 2 receptor (D2) and the serotonin transporter (5-HTT) genes produced the following result:

Likelihood of risperidone response (LoR)=[1−(−7.432+0.736A1+1.436A2+21.939B1+21.149B2−0.640C1−1.098C2)]

### Where:

A1=5-HTT 2630-T/T genotype
A2=5-HTT 2630-T/C genotype
B1=D2 Taq I A2/A2 genotype
B2=D2 Taq I A2/A1 genotype
C1=α1A Arg492/Arg492 genotype
C2=α1A Arg492/Cys genotype

The result of the logistic regression is a probability (%) of the likelihood of response or non-response. For example, a value of 0.2 will indicate a 20% chance of responding (showing an increase of at least 20 GAF score points) to treatment with risperidone.

This algorithm had the following statistic values:

Level of correct prediction: 65.2%

### PPV=63.9%

### NPV=66.7%

### Sensitivity=69.7%

### Specificity=60.6%

Classification Table^{a}

| | | | Predicted | | |
|---|---|---|---|---|---|
| | | | Risperidone | | |
| | | | response | | |
| | | | (20 points GAS) | | |
| | | | | non- | Percentage |
| Observed | | | responder | responder | Correct |
| Step 1 | Risperidone | responder | 23 | 10 | 69.7 |
| | response | non- | 13 | 20 | 60.6 |
| | (20 points | responder | | | |
| | GAS) | | | | |
| | Overall | | | | 65.2 |
| | Percentage | | | | |

^{a}The cut value is .500
cut value in table = 0.50 (i.e. the probability of a positive response of 50% was used to distinguish likely responders from likely non-responders)

Variables in the Equation

| | | B | S.E. | Wald | df | Sig. | Exp(B) |
|---|---|---|---|---|---|---|---|
| Step | a1aargcy | | | 3.082 | 2 | .214 | |
| 1^{a} | a1aargcy(1) | −.640 | .795 | .649 | 1 | .421 | .527 |
| | a1aargcy(2) | −1.098 | .627 | 3.064 | 1 | .080 | .334 |
| | d2taqa | | | 1.542 | 2 | .463 | |
| | d2taqa(1) | 21.939 | 40192.949 | .000 | 1 | 1.000 | 3.4E+09 |
| | d2taqa(2) | 21.149 | 40192.949 | .000 | 1 | 1.000 | 1.5E+09 |
| | htt18729 | | | 1.841 | 2 | .398 | |
| | htt18729(1) | .736 | 1.318 | .312 | 1 | .576 | 2.088 |
| | htt18729(2) | 1.436 | 1.381 | 1.081 | 1 | .299 | 4.203 |
| | Constant | −7.432 | 13397.650 | .000 | 1 | 1.000 | .001 |

^{a}Variable(s) entered on step 1: a1aargcy, d2taqa, htt18729.

### Example 5

An extended version of this algorithm incorporating information on genetic variants of α_{1A}, CYP2D6, 5-HT1A, 5-HT2A and 5-HT2C genes was also calculated, and produced the following result:

LoR=[1−(+11.853−22.636A1−22.231A2−1.947B1+1.415C1−0.486D1+2.513E1−0.24E2+4.623F1+1.461F2+4.71G1+0.028G2−3.989H1)]

### Where:

A1=5-HTT 2630-T/T genotype
A2=5-HTT 2630-T/C genotype
B1=D2 Taq I A2/A2 genotype
B2=D2 Taq I A2/A1 genotype
C1=α_{1A} Arg492/Arg492 genotype
C2=α_{1A} Arg492/Cys genotype
D1=α_{1A}−6274-C/C genotype
E1=CYP2D6*4 EM/EM genotype
E2=CYPD6*4 EM/PM genotype
F1=5-HT1A−1018-C/C genotype
F2=5-HT1A−1018-C/G genotype
G1=5-HT2A−1438-G/G genotype
G2=5-HT2A−1438-G/A genotype
H1=5-HT2C Cys23Ser/Cys23Ser or Cys23Ser genotypes

This algorithm had the following statistical values:

Level of prediction: 79%

### Sensitivity=72.7%

### Specificity=85.7%

### PPV=84.2%

### NPV=75%

Classification Table^{a}

| | | | Predicted | | |
|---|---|---|---|---|---|
| | | | Risperidone | | |
| | | | response | | |
| | | | (20 points GAS) | | |
| | | | | non- | Percentage |
| Observed | | | responder | responder | Correct |
| Step 1 | Risperidone | responder | 16 | 6 | 72.7 |
| | response | non- | 3 | 18 | 85.7 |
| | (20 points | responder | | | |
| | GAS) | | | | |
| | Overall | | | | 79.1 |
| | Percentage | | | | |

^{a}The cut value is .500
Note:
cut value in table = 0.50 (i.e. the probability of a positive response of 50% was used to distinguish likely responders from likely non-responders)

Variables in the Equation

| | | B | S.E. | Wald | df | Sig. | Exp(B) |
|---|---|---|---|---|---|---|---|
| Step | a1aargcy | | | .905 | 2 | .636 | |
| 1^{a} | a1aargcy(1) | 1.415 | 2.029 | .486 | 1 | .486 | 4.116 |
| | a1aargcy(2) | .000 | 1.553 | .000 | 1 | 1.000 | 1.000 |
| | d2taqa(1) | −1.947 | 1.377 | 2.001 | 1 | .157 | .143 |
| | htt18729 | | | .068 | 2 | .966 | |
| | htt18729(1) | −22.636 | 40192.978 | .000 | 1 | 1.000 | .000 |
| | htt18729(2) | −22.321 | 40192.978 | .000 | 1 | 1.000 | .000 |
| | a1a6274 | | | .107 | 1 | .744 | |
| | a1a6274(1) | −.486 | 1.487 | .107 | 1 | .744 | .615 |
| | cyp2d64 | | | 3.293 | 2 | .193 | |
| | cyp2d64(1) | 2.513 | 3.601 | .487 | 1 | .485 | 12.342 |
| | cyp2d64(2) | −.240 | 3.679 | .004 | 1 | .948 | .787 |
| | ht1a1018 | | | 3.505 | 2 | .173 | |
| | ht1a1018(1) | 4.623 | 2.469 | 3.505 | 1 | .061 | 101.776 |
| | ht1a1018(2) | 1.461 | 1.377 | 1.125 | 1 | .289 | 4.309 |
| | ht2apr | | | 6.176 | 2 | .046 | |
| | ht2apr(1) | 4.710 | 2.471 | 3.633 | 1 | .057 | 111.079 |
| | ht2apr(2) | .028 | 2.096 | .000 | 1 | .990 | 1.028 |
| | ht2cn(1) | −3.989 | 1.864 | 4.577 | 1 | .032 | .019 |
| | Constant | 11.853 | 13397.659 | .000 | 1 | .999 | 140444.3 |

^{a}Variable(s) entered on step 1: a1aargcy, d2taqa, htt18729, a1a6274, cyp2d64, ht1a1018, ht2apr, ht2cn.

### Example 6

Using a strategy encompassing a combination of information in polymorphisms/genes that had shown association with response to risperidone treatment the algorithms for the prediction of risperidone response (as measured by GAF scales) detailed in the above examples were developed.

Additional genotyping in the samples has been performed and new algorithms have been formulated as described below. The algorithms differ from the core and extended algorithms described in Examples 1−5, in the combination of polymorphisms (although there may be some polymorphisms common to both) and in the weighting given to each polymorphism.

### Materials and Methods

The mathematical algorithms were calculated using information of individual genetic association studies performed on clinical samples. After gathering information on which genetic polymorphisms may be associated with treatment variability, a mathematical algorithm was produced by logistic regression that allocates a predictive value or coefficient to each relevant genetic polymorphism according to their contribution to response variability. The prediction score was obtained by multiplying the coefficients by the genotypes present in an individual. The resulting value gives an indication of the likelihood of response.

### Clinical Samples

Risperidone clinical sample: 124 subjects (99 with schizophrenia or schizo-affective disorder, 12 with bipolar disorder, 3 with major depression and 10 with atypical psychosis) were recruited in Navarra (Northern Spain) and were of Basque and Spanish origin. All subjects were treated with the antipsychotic risperidone for a minimum of 3 months. Treatment response was assessed prospectively using the PANSS and GAF scales. Improvement of 20 points or more in the GAF scales, or at least 30% decrease in PANSS values after risperidone treatment was considered as the threshold for response or improvement in specific symptomotology (positive, negative or general).

A core algorithm (including the most reliable combination of genes) and an extended algorithm (including additional SNPs that may give an improved prediction level) determining the likelihood of general response (as measured by GAF and PANSS, two response measurement scales that measure different outcomes) have been calculated. The combination of genes used for the core algorithms is more reliable than the combination used for the extended algorithms, which are likely to change significantly when larger samples are investigated.

### Risperidone CORE Algorithm for the Prediction of General Overall Response (as Measured by Total PANSS Score)

Classification Table^{a}

| | | | Predicted | | |
|---|---|---|---|---|---|
| | | | Risperidone | | |
| | | | response | | |
| | | | (30% decrease in | | |
| | | | PANSS) | | |
| | | | | non- | Percentage |
| Observed | | | responder | responder | Correct |
| Step 1 | Risperidone | responder | 12 | 6 | 66.7 |
| | response | non- | 5 | 21 | 80.8 |
| | (30% decrease | responder | | | |
| | in PANSS) | | | | |
| | Overall | | | | 75.0 |
| | Percentage | | | | |

^{a}The cut value is .500

Variables in the Equation

| | | B | S.E. | Wald | df | Sig. | Exp(B) |
|---|---|---|---|---|---|---|---|
| Step | a1aargcy | | | 5.939 | 2 | .051 | |
| 1^{a} | a1aargcy(1) | 2.293 | 1.326 | 2.990 | 1 | .084 | 9.904 |
| | a1aargcy(2) | −.821 | .860 | .912 | 1 | .340 | .440 |
| | d2taqa(1) | 1.521 | .949 | 2.570 | 1 | .109 | 4.579 |
| | d4c521t | | | 4.185 | 2 | .123 | |
| | d4c521t(1) | −.421 | 1.215 | .120 | 1 | .729 | .656 |
| | d4c521t(2) | 1.443 | .972 | 2.202 | 1 | .138 | 4.233 |
| | Constant | −1.565 | 1.197 | 1.709 | 1 | .191 | .209 |

^{a}Variable(s) entered on step 1: a1aargcy, d2taqa, d4c521t.

LoR=[1−(−1.565+2.293A1−0.821A2+1.521B1−0.421C1+1.443C2)]

### Specificity=80.8% (21/26)

### Sensitivity=66.7% (12/18)

Positive predictive value (PPV)=70.6% (12/17)
Negative predictive value (NPV)=77.8% (21/27)
whereas:
A1=Alpha-1A adrenergic receptor rs1048101 Arg492/Arg492
A2=Alpha-1A adrenergic receptor rs1048101 Arg492/Cys
B1=Dopamine 2 (D2) rs1800497 Taq I A2/A2
C1=Dopamine 4 (D4) rs1800955−521 C/C
C2=Dopamine 4 (D4) rs1800955−521 C/T

### Risperidone CORE Algorithm for the Prediction of General Response (as Measured by GAF)

Classification Table^{a}

| | | | Predicted | | |
|---|---|---|---|---|---|
| | | | Risperidone | | |
| | | | response | | |
| | | | bimodal (GAS) | | |
| | | | | non- | Percentage |
| Observed | | | responder | responder | Correct |
| Step 1 | Risperidone | responder | 21 | 11 | 65.6 |
| | response | non- | 9 | 20 | 69.0 |
| | bimodal (GAS) | responder | | | |
| | Overall | | | | 67.2 |
| | Percentage | | | | |

^{a}The cut value is .500

Variables in the Equation

| | | B | S.E. | Wald | df | Sig. | Exp(B) |
|---|---|---|---|---|---|---|---|
| Step | a1aargcy | | | 1.845 | 2 | .397 | |
| 1^{a} | a1aargcy(1) | −.723 | .815 | .788 | 1 | .375 | .485 |
| | a1aargcy(2) | −.917 | .681 | 1.812 | 1 | .178 | .400 |
| | d2taqa(1) | .890 | .714 | 1.552 | 1 | .213 | 2.435 |
| | d4c521t | | | 6.432 | 2 | .040 | |
| | d4c521t(1) | −.961 | .941 | 1.043 | 1 | .307 | .383 |
| | d4c521t(2) | 1.057 | .643 | 2.707 | 1 | .100 | 2.878 |
| | Constant | −.615 | .937 | .431 | 1 | .512 | .541 |

^{a}Variable(s) entered on step 1: a1aargcy, d2taqa, d4c521t.

LoR=[1−(−0.615−0.723A1−0.917A2+0.890B1−0.961 C1+1.057C2)]

### Specificity=69.0%

### Sensitivity=65.6%

Positive predictive value (PPV)=70.0%
Negative predictive value (NPV)=64.5%
whereas:
A1=Alpha-1A adrenergic receptor rs1048101 Arg492/Arg492
A2=Alpha-1A adrenergic receptor rs1048101 Arg492/Cys
B1=Dopamine 2 (D2) rs1800497 Taq I A2/A2
C1=Dopamine 4 (D4) rs1800955−521 C/C
C2=Dopamine 4 (D4) rs1800955−521 C/T

### Risperidone EXTENDED Algorithm for the Prediction of General Overall Response (as Measured by Total PANSS Score)

Classification Table^{a}

| | | | Predicted | | |
|---|---|---|---|---|---|
| | | | Risperidone | | |
| | | | response | | |
| | | | (30% decrease | | |
| | | | in PANSS) | | |
| | | | | non- | Percentage |
| Observed | | | responder | responder | Correct |
| Step 1 | Risperidone | responder | 10 | 6 | 62.5 |
| | response (30% | non- | 2 | 23 | 92.0 |
| | decrease | responder | | | |
| | in PANSS) | | | | |
| | Overall | | | | 80.5 |
| | Percentage | | | | |

^{a}The cut value is .500

Variables in the Equation

| | | B | S.E. | Wald | df | Sig. | Exp(B) |
|---|---|---|---|---|---|---|---|
| Step | a1aargcy | | | 3.137 | 2 | .208 | |
| 1^{a} | a1aargcy(1) | 2.831 | 2.038 | 1.928 | 1 | .165 | 16.955 |
| | a1aargcy(2) | −.542 | 1.373 | .156 | 1 | .693 | .581 |
| | d2taqa(1) | 1.904 | 1.243 | 2.345 | 1 | .126 | 6.711 |
| | d4c521t | | | 4.083 | 2 | .130 | |
| | d4c521t(1) | −.310 | 1.539 | .041 | 1 | .840 | .733 |
| | d4c521t(2) | 2.160 | 1.345 | 2.578 | 1 | .108 | 8.671 |
| | ht1a1018 | | | 1.553 | 2 | .460 | |
| | ht1a1018(1) | 22.479 | 11698.732 | .000 | 1 | .998 | 6E+009 |
| | ht1a1018(2) | 1.680 | 1.348 | 1.553 | 1 | .213 | 5.368 |
| | PGcyp2d64 | | | .124 | 2 | .940 | |
| | PGcyp2d64(1) | −19.014 | 27726.487 | .000 | 1 | .999 | .000 |
| | PGcyp2d64(2) | .424 | 1.205 | .124 | 1 | .725 | 1.527 |
| | ht2aw | | | 2.762 | 2 | .251 | |
| | ht2aw(1) | 1.347 | 1.842 | .535 | 1 | .465 | 3.847 |
| | ht2aw(2) | 2.166 | 1.313 | 2.721 | 1 | .099 | 8.726 |
| | Constant | −5.381 | 2.939 | 3.352 | 1 | .067 | .005 |

^{a}Variable(s) entered on step 1: a1aargcy, d2taqa, d4c521t, ht1a1018, PGcyp2d64, ht2aw.

LoR=[1−(−5.381+2.831A1−0.542A2+1.904B1−0.310C1+2.160C2+22.479D1+1.68D2−19.014E1+0.424E2+1.347F1+2.166F2)]

### Specificity=92.0%

### Sensitivity=62.5%

Positive predictive value (PPV)=83.3%
Negative predictive value (NPV)=79.3%
whereas:
A1=Alpha-1A adrenergic receptor rs1048101 Arg492/Arg492
A2=Alpha-1A adrenergic receptor rs1048101 Arg492/Cys
B1=Dopamine 2 (D2) rs1800497 Taq I A2/A2
C1=Dopamine 4 (D4) rs1800955−521 C/C
C2=Dopamine 4 (D4) rs1800955−521 C/T
D1=Serotonin receptor 1A (5-HT1A) rs6295−1018 C/C
D2=Serotonin receptor 1A (5-HT1A) rs6295−1018 C/G
E1=CYP2D6*4 rs3892097 A/A
E2=CYP2D6*4 rs3892097 A/G
F1=Serotonin receptor 2A (5-HT2A) rs6313 102 T/T
F2=Serotonin receptor 2A (5-HT2A) rs6313102 T/C

### Risperidone EXTENDED Algorithm for the Prediction of General Response (as Measured by GAF)

Classification Table^{a}

| | | | Predicted | | |
|---|---|---|---|---|---|
| | | | Risperidone | | |
| | | | response | | |
| | | | bimodal | | |
| | | | (GAS) | | |
| | | | | non- | Percentage |
| Observed | | | responder | responder | Correct |
| Step 1 | Risperidone | responder | 20 | 8 | 71.4 |
| | response | non- | 7 | 20 | 74.1 |
| | bimodal (GAS) | responder | | | |
| | Overall | | | | 72.7 |
| | Percentage | | | | |

^{a}The cut value is .500

Variables in the Equation

| | | B | S.E. | Wald | df | Sig. | Exp(B) |
|---|---|---|---|---|---|---|---|
| Step | a1aargcy | | | 2.626 | 2 | .269 | |
| 1^{a} | a1aargcy(1) | −1.070 | 1.141 | .878 | 1 | .349 | .343 |
| | a1aargcy(2) | −1.494 | .922 | 2.626 | 1 | .105 | .225 |
| | d2taqa(1) | .798 | .839 | .904 | 1 | .342 | 2.220 |
| | d4c521t | | | 1.782 | 2 | .410 | |
| | d4c521t(1) | −.301 | 1.130 | .071 | 1 | .790 | .740 |
| | d4c521t(2) | .810 | .798 | 1.032 | 1 | .310 | 2.248 |
| | ht1a1018 | | | 2.975 | 2 | .226 | |
| | ht1a1018(1) | 1.982 | 1.179 | 2.828 | 1 | .093 | 7.257 |
| | ht1a1018(2) | .527 | .880 | .358 | 1 | .550 | 1.693 |
| | PGcyp2d64 | | | .252 | 2 | .882 | |
| | PGcyp2d64(1) | −21.389 | 24637.721 | .000 | 1 | .999 | .000 |
| | PGcyp2d64(2) | .409 | .814 | .252 | 1 | .616 | 1.505 |
| | ht2aw | | | 3.265 | 2 | .195 | |
| | ht2aw(1) | −2.566 | 1.423 | 3.252 | 1 | .071 | .077 |
| | ht2aw(2) | −.627 | .772 | .661 | 1 | .416 | .534 |
| | Constant | −.185 | 1.470 | .016 | 1 | .900 | .831 |

^{a}Variable(s) entered on step 1: a1aargcy, d2taqa, d4c521t, ht1a1018, PGcyp2d64, ht2aw.

LoR=[1−(−0.185−1.07A1−1.494A2+0.798B1−0.301C1+0.81C2+1.982D1+0.527D2−21.389E1+0.409E2−2.566F1−0.627F2)]

### Specificity=74.1%

### Sensitivity=71.4%

Positive predictive value (PPV)=74.1%
Negative predictive value (NPV)=71.4%
whereas:
A1=Alpha-1A adrenergic receptor rs1048101 Arg492/Arg492
A2=Alpha-1A adrenergic receptor rs1048101 Arg492/Cys
B1=Dopamine 2 (D2) rs1800497 Taq I A2/A2
C1=Dopamine 4 (D4) rs1800955−521 C/C
C2=Dopamine 4 (D4) rs1800955−521 C/T
D1=Serotonin receptor 1A (5-HT1A) rs6295−1018 C/C
D2=Serotonin receptor 1A (5-HT1A) rs6295−1018 C/G
E1=CYP2D6*4 rs3892097 A/A
E2=CYP2D6*4 rs3892097 A/G
F1=Serotonin receptor 2A (5-HT2A) rs6313 102 T/T
F2=Serotonin receptor 2A (5-HT2A) rs6313 102 T/C

Additional algorithms have been produced for the prediction of (1) improvement in positive symptoms and (2) improvement in negative symptoms, and for (3) the improvement in general psychopathology symptoms in response to risperidone.

### Risperidone Algorithm for the Prediction of Improvement in Positive Symptoms (as Measured by PANSS)

Classification Table^{a}

| | | | Predicted | | |
|---|---|---|---|---|---|
| | | | ppanssRNRri | | |
| | | | | Non- | Percentage |
| Observed | | | Responder | Responder | Correct |
| Step 1 | ppanssRNRri | Responder | 12 | 7 | 63.2 |
| | | Non- | 4 | 17 | 81.0 |
| | | Responder | | | |
| | Overall | | | | 72.5 |
| | Percentage | | | | |

^{a}The cut value is .500

Variables in the Equation

| | | B | S.E. | Wald | df | Sig. | Exp(B) |
|---|---|---|---|---|---|---|---|
| Step | comt | | | 4.453 | 2 | .108 | |
| 1^{a} | comt(1) | −3.020 | 1.544 | 3.827 | 1 | .050 | .049 |
| | comt(2) | −1.704 | 1.437 | 1.406 | 1 | .236 | .182 |
| | d2taqa(1) | .456 | .917 | .247 | 1 | .619 | 1.577 |
| | d4c521t | | | 3.452 | 2 | .178 | |
| | d4c521t(1) | 1.712 | 1.366 | 1.572 | 1 | .210 | 5.543 |
| | d4c521t(2) | 2.259 | 1.229 | 3.376 | 1 | .066 | 9.569 |
| | ht2c4757n(1) | −.638 | .845 | .569 | 1 | .451 | .528 |
| | Constant | .284 | 1.576 | .032 | 1 | .857 | 1.328 |

^{a}Variable(s) entered on step 1: comt, d2taqa, d4c521t, ht2c4757n.

LoR=[1−(0.284−3.02A1−1.704A2+0.456B1+1.712C1+2.259C2−0.638D1)]

### Specificity=81.0%

### Sensitivity=63.2%

Positive predictive value (PPV)=75.0%
Negative predictive value (NPV)=70.8%
whereas:
A1=COMT rs4680 Val158/Val158
A2=COMT rs4680 Val158/Met
B1=Dopamine 2 (D2) rs1800497 Taq I A2/A2
C1=Dopamine 4 (D4) rs1800955−521 C/C
C2=Dopamine 4 (D4) rs1800955−521 C/T
D1=Serotonin receptor 2C (5-HT2C) rs475717−145964 A/A

### Risperidone Algorithm for the Prediction of Improvement in Negative Symptoms (as Measured by PANSS)

Classification Table^{a}

| | | | Predicted | | |
|---|---|---|---|---|---|
| | | | npanssRNRri | | |
| | | | | Non- | Percentage |
| Observed | | | Responder | Responder | Correct |
| Step 1 | npanssRNRri | Responder | 2 | 9 | 18.2 |
| | | Non- | 1 | 50 | 98.0 |
| | | Responder | | | |
| | Overall | | | | 83.9 |
| | Percentage | | | | |

^{a}The cut value is .500

Variables in the Equation

| | | B | S.E. | Wald | df | Sig. | Exp(B) |
|---|---|---|---|---|---|---|---|
| Step | ht2c4757n(1) | 1.451 | .882 | 2.703 | 1 | .100 | 4.267 |
| 1^{a} | chatch6 | | | 4.689 | 2 | .096 | |
| | chatch6(1) | 3.576 | 1.652 | 4.688 | 1 | .030 | 35.739 |
| | chatch6(2) | 2.944 | 1.633 | 3.251 | 1 | .071 | 18.986 |
| | m1tsp45i | | | 1.288 | 2 | .525 | |
| | m1tsp45i(1) | −.309 | 1.862 | .028 | 1 | .868 | .734 |
| | m1tsp45i(2) | −1.170 | 1.734 | .455 | 1 | .500 | .310 |
| | nrg1533 | | | 2.640 | 2 | .267 | |
| | nrg1533(1) | −2.321 | 1.600 | 2.104 | 1 | .147 | .098 |
| | nrg1533(2) | −1.931 | 1.268 | 2.317 | 1 | .128 | .145 |
| | Constant | −.076 | 1.724 | .002 | 1 | .965 | .927 |

^{a}Variable(s) entered on step 1: ht2c4757n, chatch6, m1tsp45i, nrg1533.

LoR=[1−(−0.076+1.451A1+3.576B1+2.944B2−0.309C1−1.17C2−2.321D1−1.931D2)]

### Specificity=98.0%

### Sensitivity=18.2%

Positive predictive value (PPV)=66.7%
Negative predictive value (NPV)=84.7%
whereas:
A1=Serotonin receptor 2C (5-HT2C) rs475717−145964 A/A
B1=Choline Acetyltransferase ChAT rs1880676 G/G
B2=Choline Acetyltransferase ChAT rs1880676 G/A
C1=Muscarinic receptor M1 rs12295208−12064 T/T
C2=Muscarinic receptor M1 rs12295208−12064 T/C

### D1=Neuregulin 1 (NRG1) SNP8NRG221533 C/C

### D2=Neuregulin 1 (NRG1) SNP8NRG221533 C/T

### Risperidone Algorithm for the Prediction of Improvement in General Psychopathology Symptoms (as Measured by PANSS)

Classification Table^{a}

| | | | Predicted | | |
|---|---|---|---|---|---|
| | | | pgpanssRNRri | | |
| | | | | Non- | Percentage |
| Observed | | | Responder | Responder | Correct |
| Step 1 | pgpanssRNRri | Responder | 16 | 12 | 57.1 |
| | | Non- | 9 | 38 | 80.9 |
| | | Responder | | | |
| | Overall | | | | 72.0 |
| | Percentage | | | | |

^{a}The cut value is .500

Variables in the Equation

| | | B | S.E. | Wald | df | Sig. | Exp(B) |
|---|---|---|---|---|---|---|---|
| Step | chatch6 | | | 5.016 | 2 | .081 | |
| 1^{a} | chatch6(1) | .196 | 1.121 | .031 | 1 | .861 | 1.217 |
| | chatch6(2) | −1.053 | 1.153 | .834 | 1 | .361 | .349 |
| | ht2aw | | | 5.197 | 2 | .074 | |
| | ht2aw(1) | −1.183 | .789 | 2.251 | 1 | .134 | .306 |
| | ht2aw(2) | .407 | .673 | .367 | 1 | .545 | 1.503 |
| | nrg1533 | | | 1.693 | 2 | .429 | |
| | nrg1533(1) | 1.364 | 1.209 | 1.272 | 1 | .259 | 3.910 |
| | nrg1533(2) | .540 | .584 | .856 | 1 | .355 | 1.716 |
| | Constant | .512 | 1.162 | .195 | 1 | .659 | 1.669 |

^{a}Variable(s) entered on step 1: chatch6, ht2aw, nrg1533.
e^{β} is the estimate of the odds ratio
Sig. is statistical significance
Df is degree of freedom
Wald is the calculated coefficient
S.E. is standard error
B is the weight given to each genotype
Cut value is the threshold for response/improvement

LoR=[1−(0.512+0.196A1−1.053A2−1.183B1+0.407B2+1.364C1+0.54C2)]

### Specificity=80.9%

### Sensitivity=57.1%

Positive predictive value (PPV)=64.0%
Negative predictive value (NPV)=76.0%
whereas:
A1=Choline Acetyltransferase ChAT rs1880676 G/G
A2=Choline Acetyltransferase ChAT rs1880676 G/A
B1=Serotonin receptor 2A (5-HT2A) rs6313 102 T/T
B2=Serotonin receptor 2A (5-HT2A) rs6313 102 T/C

### C1=Neuregulin 1 (NRG1) SNP8NRG221533 C/C

### C2=Neuregulin 1 (NRG1) SNP8NRG221533 C/T

```
1. HTR1A, 5-hydroxytryptamine (serotonin) receptor 1A
LOCUS NC_000005 1269 bp DNA linear CON 30-AUG-2006
DEFINITION Homo sapiens chromosome 5, reference assembly, complete
sequence.
ACCESSION NC_000005 REGION: complement(63292034 . . . 63293302)
VERSION NC_000005.8 GI: 51511721
>ref|NC_000005.8|NC_000005: c63293302-63292034 Homo sapiens chromosome
5, reference assembly, complete sequence
ATGGATGTGCTCAGCCCTGGTCAGGGCAACAACACCACATCACCACCGGCTCCCTTTGAGACCGGCGGCA
ACACTACTGGTATCTCCGACGTGACCGTCAGCTACCAAGTGATCACCTCTCTGCTGCTGGGCACGCTCAT
CTTCTGCGCGGTGCTGGGCAATGCGTGCGTGGTGGCTGCCATCGCCTTGGAGCGCTCCCTGCAGAACGTG
GCCAATTATCTTATTGGCTCTTTGGCGGTCACCGACCTCATGGTGTCGGTGTTGGTGCTGCCCATGGCCG
CGCTGTATCAGGTGCTCAACAAGTGGACACTGGGCCAGGTAACCTGCGACCTGTTCATCGCCCTCGACGT
GCTGTGCTGCACCTCATCCATCTTGCACCTGTGCGCCATCGCGCTGGACAGGTACTGGGCCATCACGGAC
CCCATCGACTACGTGAACAAGAGGACGCCCCGGCGCGCCGCTGCGCTCATCTCGCTCACTTGGCTTATTG
GCTTCCTCATCTCTATCCCGCCCATGCTGGGCTGGCGCACCCCGGAAGACCGCTCGGACCCCGACGCATG
CACCATTAGCAAGGATCATGGCTACACTATCTATTCCACCTTTGGAGCTTTCTACATCCCGCTGCTGCTC
ATGCTGGTTCTCTATGGGCGCATATTCCGAGCTGCGCGCTTCCGCATCCGCAAGACGGTCAAAAAGGTGG
AGAAGACCGGAGCGGACACCCGCCATGGAGCATCTCCCGCCCCGCAGCCCAAGAAGAGTGTGAATGGAGA
GTCGGGGAGCAGGAACTGGAGGCTGGGCGTGGAGAGCAAGGCTGGGGGTGCTCTGTGCGCCAATGGCGCG
GTGAGGCAAGGTGACGATGGCGCCGCCCTGGAGGTGATCGAGGTGCACCGAGTGGGCAACTCCAAAGAGC
ACTTGCCTCTGCCCAGCGAGGCTGGTCCTACCCCTTGTGCCCCCGCCTCTTTCGAGAGGAAAAATGAGCG
CAACGCCGAGGCGAAGCGCAAGATGGCCCTGGCCCGAGAGAGGAAGACAGTGAAGACGCTGGGCATCATC
ATGGGCACCTTCATCCTCTGCTGGCTGCCCTTCTTCATCGTGGCTCTTGTTCTGCCCTTCTGCGAGAGCA
GCTGCCACATGCCCACCCTGTTGGGCGCCATAATCAATTGGCTGGGCTACTCCAACTCTCTGCTTAACCC
CGTCATTTACGCATACTTCAACAAGGACTTTCAAAACGCGTTTAAGAAGATCATTAAGTGTAAGTTCTGC
CGCCAGTGA
HTR2A: 5-hydroxytryptamine (serotonin) receptor 2A
LOCUS NM_000621 3009 bp mRNA linear PRI 03-DEC-2007
DEFINITION Homo sapiens 5-hydroxytryptamine (serotonin) receptor 2A
(HTR2A),
                      mRNA.
ACCESSION NM_000621
VERSION NM_000621.2 GI: 60302916
ORIGIN
>/tmp/readseq.in.25321 [Unknown form], 3009 bases, 1336 checksum.
atccagccccgggagaacagcatgtacaccagcctcagtgttacagagtg
tgggtacatcaaggtgaatggtgagcagaaactataacctgttagtcctt
ctacacctcatctgctacaagttctggcttagacatggatattctttgtg
aagaaaatacttctttgagctcaactacgaactccctaatgcaattaaat
gatgacaccaggctctacagtaatgactttaactccggagaagctaacac
ttctgatgcatttaactggacagtcgactctgaaaatcgaaccaaccttt
cctgtgaagggtgcctctcaccgtcgtgtctctccttacttcatctccag
gaaaaaaactggtctgctttactgacagccgtagtgattattctaactat
tgctggaaacatactcgtcatcatggcagtgtccctagagaaaaagctgc
agaatgccaccaactatttcctgatgtcacttgccatagctgatatgctg
ctgggtttccttgtcatgcccgtgtccatgttaaccatcctgtatgggta
ccggtggcctctgccgagcaagctttgtgcagtctggatttacctggacg
tgctcttctccacggcctccatcatgcacctctgcgccatctcgctggac
cgctacgtcgccatccagaatcccatccaccacagccgcttcaactccag
aactaaggcatttctgaaaatcattgctgtttggaccatatcagtaggta
tatccatgccaataccagtctttgggctacaggacgattcgaaggtcttt
aaggaggggagttgcttactcgccgatgataactttgtcctgatcggctc
ttttgtgtcatttttcattcccttaaccatcatggtgatcacctactttc
taactatcaagtcactccagaaagaagctactttgtgtgtaagtgatctt
ggcacacgggccaaattagcttctttcagcttcctccctcagagttcttt
gtcttcagaaaagctcttccagcggtcgatccatagggagccagggtcct
acacaggcaggaggactatgcagtccatcagcaatgagcaaaaggcatgc
aaggtgctgggcatcgtcttcttcctgtttgtggtgatgtggtgcccttt
cttcatcacaaacatcatggccgtcatctgcaaagagtcctgcaatgagg
atgtcattggggccctgctcaatgtgtttgtttggatcggttatctctct
tcagcagtcaacccactagtctacacactgttcaacaagacctataggtc
agccttttcacggtatattcagtgtcagtacaaggaaaacaaaaaaccat
tgcagttaattttagtgaacacaataccggctttggcctacaagtctagc
caacttcaaatgggacaaaaaaagaattcaaagcaagatgccaagacaac
agataatgactgctcaatggttgctctaggaaagcagcattctgaagagg
cttctaaagacaatagcgacggagtgaatgaaaaggtgagctgtgtgtga
taggctagttgccgtggcaactgtggaaggcacactgagcaagttttcac
ctatctggaaaaaaaaaaatatgagattggaaaaaattagacaagtctag
tggaaccaacgatcatatctgtatgcctcattttattctgtcaatgaaaa
gcggggttcaatgctacaaaatgtgtgcttggaaaatgttctgacagcat
ttcagctgtgagctttctgatacttatttataacattgtaaatgatatgt
ctttaaaatgattcacttttattgtataattatgaagccctaagtaaatc
taaattaacttctattttcaagtggaaaccttgctgctatgctgttcatt
gatgacatgggattgagttggttacctattgctgtaaataaaaatagcta
taaatagtgaaaattttattgaatataatggcctcttaaaaattatcttt
aaaacttactatggtatatattttgaaaggagaaaaaaaaagccactaag
gtcagtgttataaaatctgtattgctaagataattaaatgaaatacttga
caacatttttcattcctgctttttcatagataccattttgaaatattcac
aaggttgctggcatttgctgcatttcaagttaattctcagaagtgaaaaa
gacttcaaatgttattcaataactattgctgctttctcttctacttcttg
tgctttactctgaatttccagtgtggtcttgtttaatatttgttcctcta
ggtaaactagcaaaaggatgatttaacattaccaaatgcctttctagcaa
ttgcttctctaaaacagcactatcgaggtatttggtaacttgctgtgaaa
tgactgcatcatgcatgcactcttttgagcagtaaatgtatattgatgta
actgtgtcaggattgaggatgaactcaggtttccggctactgacagtggt
agagtcctaggacatctctgtaaaaagcaggtgactttcctatgacactc
atcaggtaaactgatgctttcagatccatcggtttatactatttattaaa
accattctgcttggttccacaatcatctattgagtgtacatttatgtgtg
aagcaaatttctagatatgagaaatataaaaataattaaaacaaaatcct
tgccttcaaacgaaatggctcggccaggcacggaggctcgtgcatgtaat
cctagcactttgggaggctgagatgggaggatcacttgaggccaagagtt
tgagaccaacctgggtaacaaagtgagacctccctgtctctacaaaaaaa
atcaaaaaattatctgatccttgtggcacacaactgtggtcccagctaca
ggggaggctgagacgcaaggatcacttgagcccagaagctcaaggctgca
gtgagccaagttcacaccactgccatttcctcctgggcaacagagtgaga
ccctatcac
HTR2C: 5-hydroxytryptamine (serotonin) receptor 2C
NM_000868 4775 bp mRNA linear PRI 09-DEC-2007
DEFINITION Homo sapiens 5-hydroxytryptamine (serotonin) receptor 2C
(HTR2C),
                      mRNA.
ACCESSION NM_000868
VERSION NM_000868.1 GI: 4504540
>/tmp/readseq.in.25345 [Unknown form], 4775 bases, 171B checksum.
acccgcgcgaggtaggcgctctggtgcttgcggaggacgcttccttcctc
agatgcaccgatcttcccgatactgcctttggagcggctagattgctagc
cttggctgctccattggcctgccttgccccttacctgccgattgcatatg
aactcttcttctgtctgtacatcgttgtcgtcggagtcgtcgcgatcgtc
gtggcgctcgtgtgatggccttcgtccgtttagagtagtgtagttagtta
ggggccaacgaagaagaaagaagacgcgattagtgcagagatgctggagg
tggtcagttactaagctagagtaagatagcggagcgaaaagagccaaacc
tagccggggggcgcacggtcacccaaaggaggtcgactcgccggcgcttc
ctatcgcgccgagctccctccattcctctccctccgccgaggcgcgaggt
tgcggcgcgcagcgcagcgcagctcagcgcaccgactgccgcgggctccg
ctgggcgattgcagccgagtccgtttctcgtctagctgccgccgcggcga
ccgctgcctggtcttcctcccggacgctagtgggttatcagctaacaccc
gcgagcatctataacataggccaactgacgccatccttcaaaaacaacta
aaggatgatatgatgaacctagcctgttaatttcgtcttctcaattttaa
actttggttgcttaagactgaagcaatcatggtgaacctgaggaatgcgg
tgcattcattccttgtgcacctaattggcctattggtttggcaatgtgat
atttctgtgagcccagtagcagctatagtaactgacattttcaatacctc
cgatggtggacgcttcaaattcccagacggggtacaaaactggccagcac
tttcaatcgtcatcataataatcatgacaataggtggcaacatccttgtg
atcatggcagtaagcatggaaaagaaactgcacaatgccaccaattactt
cttaatgtccctagccattgctgatatgctagtgggactacttgtcatgc
ccctgtctctcctggcaatcctttatgattatgtctggccactacctaga
tatttgtgccccgtctggatttctttagatgttttattttcaacagcgtc
catcatgcacctctgcgctatatcgctggatcggtatgtagcaatacgta
atcctattgagcatagccgtttcaattcgcggactaaggccatcatgaag
attgctattgtttgggcaatttctataggtgtatcagttcctatccctgt
gattggactgagggacgaagaaaaggtgttcgtgaacaacacgacgtgcg
tgctcaacgacccaaatttcgttcttattgggtccttcgtagctttcttc
ataccgctgacgattatggtgattacgtattgcctgaccatctacgttct
gcgccgacaagctttgatgttactgcacggccacaccgaggaaccgcctg
gactaagtctggatttcctgaagtgctgcaagaggaatacggccgaggaa
gagaactctgcaaaccctaaccaagaccagaacgcacgccgaagaaagaa
gaaggagagacgtcctaggggcaccatgcaggctatcaacaatgaaagaa
aagcttcgaaagtccttgggattgttttctttgtgtttctgatcatgtgg
tgcccatttttcattaccaatattctgtctgttctttgtgagaagtcctg
taaccaaaagctcatggaaaagcttctgaatgtgtttgtttggattggct
atgtttgttcaggaatcaatcctctggtgtatactctgttcaacaaaatt
taccgaagggcattctccaactatttgcgttgcaattataaggtagagaa
aaagcctcctgtcaggcagattccaagagttgccgccactgctttgtctg
ggagggagcttaatgttaacatttatcggcataccaatgaaccggtgatc
gagaaagccagtgacaatgagcccggtatagagatgcaagttgagaattt
agagttaccagtaaatccctccagtgtggttagcgaaaggattagcagtg
tgtgagaaagaacagcacagtcttttcctacggtacaagctacatatgta
ggaaaattttcttctttaatttttctgttggtcttaactaatgtaaatat
tgctgtctgaaaaagtgtttttacatatagctttgcaaccttgtacttta
caatcatgcctacattagtgagatttagggttctatatttactgtttata
ataggtggagactaacttattttgattgtttgatgaataaaatgtttatt
tttgctctccctcccttctttccttccttttttcctttcttccttccttt
ctctctttcttttgtgcatatggcaacgttcatgttcatctcaggtggca
tttgcaggtgaccagaatgaggcacatgacagtggttatatttcaaccac
acctaaattaacaaattcagtggacatttgttctgggttaacagtaaata
tacactttacattcttgctctgctcatctacacatataaacacagtaaga
taggttctgctttctgatacatctgtcagtgagtcagaggcagaacctag
tcttgttgttcatataggggcaaaaatttgacattgtcagaatgttgtgt
tggtatttactgcaatgtctgtccctaaacatagtggtattttaacatag
cagctggttaaccgggactacagaagtggaaggataatgagatgtaatac
accaaatagcttttcacttcttaaggacagtgttcaaattctgattatta
caacaagcaaactgaaattagtgttttcattctggtccttagtaaattcc
taattctatgattaaactgggaaatgagatcccagagttatttcccaacc
caggattcaacatcaattgggttttgatctcagcatcctggaaatttgtg
tgcttcacacaaagtgaaattagtattttgagccttattaaaatattttc
ttaattatggtacctctgtctataggacttaatttagcagtccatttttg
agtaaaacttgtattggaagtatagatggtagaaactttggaagttttac
ttgattaaggactacagaattgggcccttagaatgtgaaaaaaaaaagta
attaaaaagacacttttaccgaactcgggattacagaaacacggagtttc
catttggattttaaacaaaatttatgtcattttcagatccttccaaactc
tctagtgcaggaaaaggctgcagctaatttgtgaaagtggcaagctcttc
attgcactgcagttatttaccagaagtttaaatctttgttaaaatatagt
gttgtgttacaataagtgttggccatcatttcattcgtgggcctgctgct
ctctaagaattcagtagcattttaatagtttctaaaccatgaaaagtttt
caagcattgctaaagtcaggccattcagtctatgctgtgtgcagagtata
caagtgtttctagtaacagtatttccatacgtgcccatttcacacaactg
tggataaattttggaagaattcatgatgctagttcttacgcttgacagtt
acttacacacctgagaatgtgcctctcagtatcttaaaattggttaatga
aaaatctgaatttctaaaacccttggtctgtgttctcaacacacagtata
gataaatccaatagtctgccacaagggcagtggaagagctgctgtatttg
aggaaactcatacagtctctatttgatttgcaacactggccaaacatcag
tcatttgcttgagcatgcccaaatattacatgaaagtcaagtctacctgc
cttgcctgttaggtctgttgaagtgcatgttaaaataattatatgaagca
gaatgagatgatttaattcttaccgaaatgaaaatggctgaagaaacaca
gcatgcatttagcatgagttctgcacatacagatggtgtcctgcatgtat
gccatgtatgttgcatgaatccatcgatttgtattaatgtagggcagaat
agctgatagaagaaggactgaagaaaatccttcagcaatccttaaaaaga
ccatgcattcagatctgaagtagtgtgagtgttagaaaaaactggaaaca
tctgatttctgaactatcagggcaagctcatagcacatgttttacaaaga
aacaaaatataaatcacagatttccaaaagtactagcaataagttgaatg
ataatagctcacagcacatttgttaatgattcttgtgtcatcaagtagta
gtacttaatagtacccaacctggtaattatcctcaagttgtgtgctattc
gtaagttctgtgcagtttggtatgaaacaaatatactcatttggatataa
atcttacccttcaatgttaaatctacaaacttttataaatgttttaaaga
agtccatgtgataattgtaaaggtgatgaatttaccatcaaacaaatcat
tttgatgtattattatatatgtatatctgtgtaagacacgtgcaacagac
tgccttatattattttctgtaattcttctcctttgtcaaatggtattttt
tgtgaatggttgcaaagtgttgtcttattcctaattcctgtatgttatcc
actacaggttttatgagacttcctattaatttattaaatttattaaatgt
tgaaaaaaaaaaaaaaaaaaaaaaa
SLC6A4 solute carrier family 6 (neurotransmitter transporter, serotonin), member 4
(5-HTT)
LOCUS NM—001045 2775 bp mRNA linear PRI 09-DEC-2007
DEFINITION Homo sapiens solute carrier family 6 (neurotransmitter
transporter.
                      serotonin), member 4 (SLC6A4), mRNA.
ACCESSION NM—001045
VERSION NM—001045.3 GI: 145553964
>/tmp/readseq.in.25354 (Unknown form), 2775 bases, 905 checksum.
acagccagcgccgccgggtgcctcgagggcgcgaggccagcccgcctgcc
cagcccgggaccagcctccccgcgcagcctggcaggtctcctggaggcaa
ggcgaccttgcttgccctctcttgcagaataacaaggggcttagccacag
gagttgctggcaagtggaaagaagaacaaatgagtcaatcccgacgtgtc
aatcccgacgatagagagctcggaggtgatccacaaatccaagcacccag
agatcaattgggatccttggcagatggacatcagtgtcatttactaacca
gcaggatggagacgacgcccttgaattctcagaagcagctatcagcgtgt
gaagatggagaagattgtcaggaaaacggagttctacagaaggttgttcc
caccccaggggacaaagtggagtccgggcaaatatccaatgggtactcag
cagttccaagtcctggtgcgggagatgacacacggcactctatcccagcg
accaccaccaccctagtggctgagcttcatcaaggggaacgggagacctg
gggcaagaaggtggatttccttctctcagtgattggctatgctgtggacc
tgggcaatgtctggcgcttcccctacatatgttaccagaatggagggggg
gcattcctcctcccctacaccatcatggccatttttgggggaatcccgct
cttttacatggagctcgcactgggacagtaccaccgaaatggatgcattt
caatatggaggaaaatctgcccgattttcaaagggattggttatgccatc
tgcatcattgccttttacattgcttcctactacaacaccatcatggcctg
ggcgctatactacctcatctcctccttcacggaccagctgccctggacca
gctgcaagaactcctggaacactggcaactgcaccaattacttctccgag
gacaacatcacctggaccctccattccacgtcccctgctgaagaatttta
cacgcgccacgtcctgcagatccaccggtctaaggggctccaggacctgg
ggggcatcagctggcagctggccctctgcatcatgctgatcttcactgtt
atctacttcagcatctggaaaggcgtcaagacctctggcaaggtggtgtg
ggtgacagccaccttcccttatatcatcctttctgtcctgctggtgaggg
gtgccaccctccctggagcctggaggggtgttctcttctacttgaaaccc
aattggcagaaactcctggagacaggggtgtggatagatgcagccgctca
gatcttcttctctcttggtccgggctttggggtcctgctggcttttgcta
gctacaacaagttcaacaacaactgctaccaagatgccctggtgaccagc
gtggtgaactgcatgacgagcttcgtttcgggatttgtcatcttcacagt
gctcggttacatggctgagatgaggaatgaagatgtgtctgaggtggcca
aagacgcaggtcccagcctcctcttcatcacgtatgcagaagcgatagcc
aacatgccagcgtccactttctttgccatcatcttctttctgatgttaat
cacgctgggcttggacagcacgtttgcaggcttggagggggtgatcacgg
ctgtgctggatgagttcccacacgtctgggccaagcgccgggagcggttc
gtgctcgccgtggtcatcacctgcttctttggatccctggtcaccctgac
ttttggaggggcctacgcggtgaagctgctggaggagtatgccacggggc
ccgcagtgctcactgtcgcgctgatcgaagcagtcgctgtgtcttggttc
tatggcatcactcagttctgcagggacgtgaaggaaatgctcggcttcag
cccggggtggttctggaggatctgctgggtggccatcagccctctgtttc
tcctgttcatcatttgcagttttctgatgagcccgccacaactacgactt
ctccaatataattatccttactggagtatcatcttgggttactgcatagg
aacctcatctttcatttgcatccccacatatatagcttatcggttgatca
tcactccagggacatttaaagagcgtattattaaaagtattaccccagaa
acaccaacagaaattccttgtggggacatccgcttgaatgctgtgtaaca
cactcaccgagaggaaaaaggcttctccacaacctcctcctccagttctg
atgaggcacgcctgccttctcccctccaagtgaatgagtttccagctaag
cctgatgatggaagggccttctccacagggacacagtctggtgcccagac
tcaaggcctccagccacttatttccatggattcccctggacatattccca
tggtagactgtgacacagctgagctggcctattttggacgtgtgaggatg
tggatggaggtgatgaaaaccaccctatcatcagttaggattaggtttag
aatcaagtctgtgaaagtctcctgtatcatttcttggtatgatcattggt
atctgatatctgtttgcttctaaaggtttcactgttcatgaatacgtaaa
ctgcgtaggagagaacagggatgctatctcgctagccatatattttctga
gtagcatatataattttattgctggaatctactagaaccttctaatccat
gtgctgctgtggcatcaggaaaggaagatgtaagaagctaaaatgaaaaa
tagtgtgtccatgcaaaaaaaaaaa
(alpha 1A ) ADRA1A adrenergic, alpha-1A-, receptor
LOCUS NM_033303 2304 bp mRNA linear PRI 03-DEC-2007
DEFINITION Homo sapiens adrenergic, alpha-1A-, receptor (ADRA1A),
transcript
                      variant 2, mRNA.
ACCESSION NM_033303
VERSION NM_033303.3 GI: 111118985
>/tmp/readseq.in.25374 [Unknown form], 2318 bases, 23CF checksum.
gaattccgaatcatgtgcagaatgctgaatcttcccccagccaggacgaa
taagacagcgcggaaaagcagattctcgtaattctggaattgcatgttgc
aaggagtctcctggatcttcgcacccagcttcgggtagggagggagtccg
ggtcccgggctaggccagcccggcaggtggagagggtccccggcagcccc
gcgcgcccctggccatgtctttaatgccctgccccttcatgtggccttct
gagggttcccagggctggccagggttgtttcccacccgcgcgcgcgctct
cacccccagccaaacccacctggcagggctccctccagccgagacctttt
gattcccggctcccgcgctcccgcctccgcgccagcccgggaggtggccc
tggacagccggacctcgcccggccccggctgggaccatggtgtttctctc
gggaaatgcttccgacagctccaactgcacccaaccgccggcaccggtga
acatttccaaggccattctgctcggggtgatcttggggggcctcattctt
ttcggggtgctgggtaacatcctagtgatcctctccgtagcctgtcaccg
acacctgcactcagtcacgcactactacatcgtcaacctggcggtggccg
acctcctgctcacctccacggtgctgcccttctccgccatcttcgaggtc
ctaggctactgggccttcggcagggtcttctgcaacatctgggcggcagt
ggatgtgctgtgctgcaccgcgtccatcatgggcctctgcatcatctcca
tcgaccgctacatcggcgtgagctacccgctgcgctacccaaccatcgtc
acccagaggaggggtctcatggctctgctctgcgtctgggcactctccct
ggtcatatccattggacccctgttcggctggaggcagccggcccccgagg
acgagaccatctgccagatcaacgaggagccgggctacgtgctcttctca
gcgctgggctccttctacctgcctctggccatcatcctggtcatgtactg
ccgcgtctacgtggtggccaagagggagagccggggcctcaagtctggcc
tcaagaccgacaagtcggactcggagcaagtgacgctccgcatccatcgg
aaaaacgccccggcaggaggcagcgggatggccagcgccaagaccaagac
gcacttctcagtgaggctcctcaagttctcccgggagaagaaagcggcca
aaacgctgggcatcgtggtcggctgcttcgtcctctgctggctgcctttt
ttcttagtcatgcccattgggtctttcttccctgatttcaagccctctga
aacagtttttaaaatagtattttggctcggatatctaaacagctgcatca
accccatcatatacccatgctccagccaagagttcaaaaaggcctttcag
aatgtcttgagaatccagtgtctctgcagaaagcagtcttccaaacatgc
cctgggctacaccctgcacccgcccagccaggccgtggaagggcaacaca
aggacatggtgcgcatccccgtgggatcaagagagaccttctacaggatc
tccaagacggatggcgtttgtgaatggaaatttttctcttccatgccccg
tggatctgccaggattacagtgtccaaagaccaatcctcctgtaccacag
cccggacgaagtctcgctctgtcaccaggctggagtgcagtggcatgatc
ttggctcactgcaacctccgcctcccgggttcaagagattctcctgcctc
agcctcccaagcagctgggactacagggatgtgccaccaggccgacgcca
ccaggcccagctaatttttgtatttttagtagagacggggtttcaccatg
ttggccaggatgatctcgatctcttgacctcatgatctgcctgcctcagc
ctcccaaagtgctgggattacaggcgtgagccaccgtgcccggcccaact
attttttttttttatcttttttaacagtgcaatcctttctgtggatgaaa
tcttgctcagaagctcaatatgcaaaagaaagaaaaacagcagggctgga
cggatgttgggagtggggtaagaccccaaccactcagaaccaccccccca
acacacacacacattctctccatggtgactggtgaggggcctctagaggg
tacatagtacaccatggagcacggtttaagcaccactggactacacattc
ttctgtggcagttatcttaccttcccatagacacccagcccatagccatt
ggtt-@T-->Cnotctot
CYP2D6: cytochrome P450, family 2, subfamily D, polypeptide 6,
HUMCYPDB1 1567 bp mRNA linear PRI 02-NOV-1994
DEFINITION Human cytochrome P450 dbl mRNA, complete cds.
ACCESSION M20403 M19697
VERSION M20403.1 GI: 181349
>/tmp/readseq.in.25381 [Unknown form], 1567 bases, 16F6 checksum.
atggggctagaagcactggtgcccctggccgtgatagtggccatcttcct
gctcctggtggacctgatgcaccggcgccaacgctgggctgcacgctacc
caccaggccccctgccactgcccgggctgggcaacctgctgcatgtggac
ttccagaacacaccatactgcttcgaccagttgcggcgccgcttcgggga
cgtgttcagcctgcagctggcctggacgccggtggtcgtgctcaatgggc
tggcggccgtgcgcgaggcgctggtgacccacggcgaggacaccgccgac
cgcccgcctgtgcccatcacccagatcctgggtttcgggccgcgttccca
aggggtgttcctggcgcgctatgggcccgcgtggcgcgagcagaggcgct
tctccgtgtccaccttgcgcaacttgggcctgggcaagaagtcgctggag
cagtgggtgaccgaggaggccgcctgcctttgtgccgccttcgccaacca
ctccggacgcccctttcgccccaacggtctcttggacaaagccgtgagca
acgtgatcgcctccctcacctgcgggcgccgcttcgagtacgacgaccct
cgcttcctcaggctgctggacctagctcaggagggactgaaggaggagtc
gggctttctgcgcgaggtgctgaatgctgtccccgtcctcctgcatatcc
cagcgctggctggcaaggtcctacgcttccaaaaggctttcctgacccag
ctggatgagctgctaactgagcacaggatgacctgggacccagcccagcc
cccccgagacctgactgaggccttcctggcagagatggagaaggccaagg
ggaaccctgagagcagcttcaatgatgagaacctgcgcatagtggtggct
gacctgttctctgccgggatggtgaccacctcgaccacgctggcctgggg
cctcctgctcatgatcctacatccggatgtgcagcgccgtgtccaacagg
agatcgacgacgtgatagggcaggtgcggcgaccagagatgggtgaccag
gctcacatgccctacaccactgccgtgattcatgaggtgcagcgctttgg
ggacatcgtccccctgggtatgacccatatgacatcccgtgacatCgaag
tacagggcttccgcatccctaagggaacgacactcatcaccaacctgtca
tcggtgctgaaggatgaggccgtctgggagaagcccttccgcttccaccc
cgaacacttcctggatgcccagggccactttgtgaagccggaggccttcc
tgcctttctcagcaggccgccgtgcatgcctcggggagcccctggcccgc
atggagctcttcctcttcttcacctccctgctgcagcacttcagcttctc
ggtgcccactggacagccccggcccagccaccatggtgtctttgctttcc
tggtgagcccatccccctatgagctttgtgctgtgccccgctagaatggg
gtacctagtccccagcctgctcctagcccagaggctctaatgtacaataa
agcaatgtggtagttcc
DRD2: dopamine receptor D2
NM_000795 2643 bp mRNA linear PRI 09-DEC-2007
DEFINITION Homo sapiens dopamine receptor D2 (DRD2), transcript
variant 1,
                      mRNA.
ACCESSION NM_000795
VERSION NM_000795.2 GI: 17986271
>/tmp/readseq.in.25386 [Unknown form], 2643 bases, 1D9B checksum.
ggcagccgtccggggccgccactctcctcggccggtccctggctcccgga
ggcggccgcgcgtggatgcggcgggagctggaagcctcaagcagccggcg
ccgtctctgccccggggcgccctatggcttgaagagcctggccacccagt
ggctccaccgccctgatggatccactgaatctgtcctggtatgatgatga
tctggagaggcagaactggagccggcccttcaacgggtcagacgggaagg
cggacagaccccactacaactactatgccacactgctcaccctgctcatc
gctgtcatcgtcttcggcaacgtgctggtgtgcatggctgtgtcccgcga
gaaggcgctgcagaccaccaccaactacctgatcgtcagcctcgcagtgg
ccgacctcctcgtcgccacactggtcatgccctgggttgtctacctggag
gtggtaggtgagtggaaattcagcaggattcactgtgacatcttcgtcac
tctggacgtcatgatgtgcacggcgagcatcctgaacttgtgtgccatca
gcatcgacaggtacacagctgtggccatgcccatgctgtacaatacgcgc
tacagctccaagcgccgggtcaccgtcatgatctccatcgtctgggtcct
gtccttcaccatctcctgcccactcctcttcggactcaataacgcagacc
agaacgagtgcatcattgccaacccggccttcgtggtctactcctccatc
gtctccttctacgtgcccttcattgtcaccctgctggtctacatcaagat
ctacattgtcctccgcagacgccgcaagcgagtcaacaccaaacgcagca
gccgagctttcagggcccacctgagggctccactaaagggcaactgtact
caccccgaggacatgaaactctgcaccgttatcatgaagtctaatgggag
tttcccagtgaacaggcggagagtggaggctgcccggcgagcccaggagc
tggagatggagatgctctccagcaccagcccacccgagaggacccggtac
agccccatcccacccagccaccaccagctgactctccccgacccgtccca
ccatggtctccacagcactcccgacagccccgccaaaccagagaagaatg
ggcatgccaaagaccaccccaagattgccaagatctttgagatccagacc
atgcccaatggcaaaacccggacctccctcaagaccatgagccgtaggaa
gctctcccagcagaaggagaagaaagccactcagatgctcgccattgttc
tcggcgtgttcatcatctgctggctgcccttcttcatcacacacatcctg
aacatacactgtgactgcaacatcccgcctgtcctgtacagcgccttcac
gtggctgggctatgtcaacagcgccgtgaaccccatcatctacaccacct
tcaacattgagttccgcaaggccttcctgaagatcctccactgctgactc
tgctgcctgcccgcacagcagcctgcttcccacctccctgcccaggccgg
ccagcctcacccttgcgaaccgtgagcaggaaggcctgggtggatcggcc
tcctcttcaccccggcaggccctgcagtgttcgcttggctccatgctcct
cactgcccgcacaccctcactctgccagggcagtgctagtgagctgggca
tggtaccagccctggggctgggccccccagctcaggggcagctcatagag
tcccccctcccacctccagtccccctatccttggcaccaaagatgcagcc
gccttccttgaccttcctctggggctctagggttgctggagcctgagtca
gggcccagaggctgagttttctctttgtggggcttggcgtggagcaggcg
gtggggagagatggacagttcacaccctgcaaggcccacaggaggcaagc
aagctctcttgccgaggagccaggcaacttcagtcctgggagacccatgt
aaataccagactgcaggttggaccccagagattcccaagccaaaaacctt
agctccctcccgcaccccgatgtggacctctactttccaggctagtccgg
acccacctcaccccgttacagctccccaagtggtttccacatgctctgag
aagaggagccctcatcttgaagggcccaggagggtctatggggagaggaa
ctccttggcctagcccaccctgctgccttctgacggccctgcaatgtatc
ccttctcacagcacatgctggccagcctggggcctggcagggaggtcagg
ccctggaactctatctgggcctgggctaggggacatcagaggttctttga
gggactgcctctgccacactctgacgcaaaaccactttccttttctattc
cttctggcctttcctctctcctgtttcccttcccttccactgcctctgcc
ttagaggagcccacggctaagaggctgctgaaaaccatctggcctggcct
ggccctgccctgaggaaggaggggaagctgcagcttgggagagcccctgg
ggcctagactctgtaacatcactatccatgcaccaaactaataaaacttt
gacgagtcaccttccaggacccctgggtaaaaaaaaaaaaaaa
DRD4: dopamine receptor DA:
LOCUS NM_000797 1360 bp mRNA linear PRI 09-DEC-2007
DEFINITION Homo sapiens dopamine receptor D4 (DRD4), mRNA.
ACCESSION NM_000797
VERSION NM_000797.2 GI: 32483396
>/tmp/readseq.in.25390 [Unknown form], 1360 bases, 123F checksum.
atggggaaccgcagcaccgcggacgcggacgggctgctggctgggcgcgg
gccggccgcgggggcatctgcgggggcatctgcggggctggctgggcagg
gcgcggcggcgctggtggggggcgtgccgctcatcggcgcggtgctcgcg
gggaactcgctcgtgtgcgtgagcgtggccaccgagcgcgccctgcagac
gcccaccaactccttcatcgtgagcctggcggccgccgacctcctcctcg
ctctcctggtgctgccgctcttcgtctactccgaggtccagggtggcgcg
tggctgctgagcccccgcctgtgcgacgccctcatggccatggacgtcat
gctgtgcaccgcctccatcttcaacctgtgcgccatcagcgtggacaggt
tcgtggccgtggccgtgccgctgcgctacaaccggcagggtgggagccgc
cggcagctgctgctcatcggcgccacgtggctgctgtccgcggcggtggc
ggcgcccgtactgtgcggcctcaacgacgtgcgcggccgcgaccccgccg
tgtgccgcctggaggaccgcgactacgtggtctactcgtccgtgtgctcc
ttcttcctaccctgcccgctcatgctgctgctctactgggccacgttccg
cggcctgcagcgctgggaggtggcacgtcgcgccaagctgcacggccgcg
cgccccgccgacccagcggccctggcccgccttcccccacgccacccgcg
ccccgcctcccccaggacccctgcggccccgactgtgcgccccccgcgcc
cggccttccccggggtccctgcggccccgactgtgcgcccgccgcgccca
gcctcccccaggacccctgtggccccgactgtgcgccccccgcgcccggc
ctccccccggacccctgcggctccaactgtgctccccccgacgccgtcag
agccgccgcgctcccaccccagactccaccgcagacccgcaggaggcggc
gtgccaagatcaccggccgggagcgcaaggccatgagggtcctgccggtg
gtggtcggggccttcctgctgtgctggacgcccttcttcgtggtgcacat
cacgcaggcgctgtgtcctgcctgctccgtgcccccgcggctggtcagcg
ccgtcacctggctgggctacgtcaacagcgccctcaaccccgtcatctac
actgtcttcaacgccgagttccgcaacgtcttccgcaaggccctgcgtgc
ctgctgctgagccgggcacccccggacgccccccggcctgatggccaggc
ctcagggaccaaggagatggggagggcgcttttgtacgttaattaaacaa
attccttccc
COMT: catechol-O-methyltransferase:
NM_007310 1067 bp mRNA linear PRI 09-DEC-2007
DEFINITION Homo sapiens catechol-O-methyltransferase (COMT),
transcript
                      variant S-COMT, mRNA.
ACCESSION NM_007310
VERSION NM_007310.1 GI: 6466449
>/tmp/readseq.in.25398 [Unknown form], 1067 bases, 1024 checksum.
gctgttggcagctgtgttgctgggcctggtgctgctggtggtgctgctgc
tgcttctgaggcactggggctggggcctgtgccttatcggctggaacgag
ttcatcctgcagcccatccacaacctgctcatgggtgacaccaaggagca
gcgcatcctgaaccacgtgctgcagcatgcggagcccgggaacgcacaga
gcgtgctggaggccattgacacctactgcgagcagaaggagtgggccatg
aacgtgggcgacaagaaaggcaagatcgtggacgccgtgattcaggagca
ccagccctccgtgctgctggagctgggggcctactgtggctactcagctg
tgcgcatggcccgcctgctgtcaccaggggcgaggctcatcaccatcgag
atcaaccccgactgtgccgccatcacccagcggatggtggatttcgctgg
cgtgaaggacaaggtcacccttgtggttggagcgtcccaggacatcatcc
cccagctgaagaagaagtatgatgtggacacactggacatggtcttcctc
gaccactggaaggaccggtacctgccggacacgcttctcttggaggaatg
tggcctgctgcggaaggggacagtgctactggctgacaacgtgatctgcc
caggtgcgccagacttcctagcacacgtgcgcgggagcagctgctttgag
tgcacacactaccaatcgttcctggaatacagggaggtggtggacggcct
ggagaaggccatctacaagggcccaggcagcgaagcagggccctgactgc
ccccccggcccccctctcgggctctctcacccagcctggtactgaaggtg
ccagacgtgctcctgctgaccttctgcggctccgggctgtgtcctaaatg
caaagcacacctcggccgaggcctgcgccctgacatgctaacctctctga
actgcaacactggattgttcttttttaagactcaatcatgacttctttac
taacactggctagctatattatcttatatactaatatcatgttttaaaaa
tataaaatagaaattaa
(M1) CHRM1: cholinergic receptor, muscarinic 1:
NM_000738 2863 bp mRNA linear PRI 25-SEP-2007
DEFINITION Homo sapiens cholinergic receptor, muscarinic 1 (CHRM1),
mRNA.
ACCESSION NM_000738
VERSION NM_000738.2 GI: 37622909
>/tmp/readseq.in.25401 [Unknown form], 2863 bases, A5 checksum.
tggggctcaaattgggtgccctggtgaaggaggggggcacactccagaac
ctagtccaaccccagacgctgcctgaggctcccctccagctcccctccct
tccttttctccctttcctccctccctctctttccctttctccctccccgc
taaggctggcgtgccagggggtgggacatgccaatcactggctgtgcctc
tcccgctgccagcacagggcgcagctccccctgggagccaggtgtttggg
tccctggagacgccgcaggcccccagggaggcagtggggctgaggaccct
acagacccctcttcagccccgtggtgatgactttcccctgaggaagccct
gtagcgtgcctggaggaaggggctctccaaccccagccccacctagccac
catgaacacttcagccccacctgctgtcagccccaacatcaccgtcctgg
caccaggaaagggtccctggcaagtggccttcattgggatcaccacgggc
ctcctgtcgctagccacagtgacaggcaacctgctggtactcatctcttt
caaggtcaacacggagctcaagacagtcaataactacttcctgctgagcc
tggcctgtgctgacctcatcatcggtaccttctccatgaacctctatacc
acgtacctgctcatgggccactgggctctgggcacgctggcttgtgacct
ctggctggccctggactatgtggccagcaatgcctccgtcatgaatctgc
tgctcatcagctttgaccgctacttctccgtgactcggcccctgagctac
cgtgccaagcgcacaccccgccgggcagctctgatgatcggcctggcctg
gctggtttcctttgtgctctgggccccagccatcctcttctggcagtacc
tggtaggggagcggacagtgctagctgggcagtgctacatccagttcctc
tcccagcccatcatcacctttggcacagccatggctgccttctacctccc
tgtcacagtcatgtgcacgctctactggcgcatctaccgggagacagaga
accgagcacgggagctggcagcccttcagggctccgagacgccaggcaaa
gggggtggcagcagcagcagctcagagaggtctcagccaggggctgaggg
ctcaccagagactcctccaggccgctgctgtcgctgctgccgggccccca
ggctgctgcaggcctacagctggaaggaagaagaggaagaggacgaaggc
tccatggagtccctcacatcctcagagggagaggagcctggctccgaagt
ggtgatcaagatgccaatggtggaccccgaggcacaggcccccaccaagc
agcccccacggagctccccaaatacagtcaagaggccgactaagaaaggg
cgtgatcgagctggcaagggccagaagccccgtggaaaggagcagctggc
caagcggaagaccttctcgctggtcaaggagaagaaggcggctcggaccc
tgagtgccatcctcctggccttcatcctcacctggacaccgtacaacatc
atggtgctggtgtccaccttctgcaaggactgtgttcccgagaccctgtg
ggagctgggctactggctgtgctacgtcaacagcaccatcaaccccatgt
gctacgcactctgcaacaaagccttccgggacacctttcgcctgctgctg
ctttgccgctgggacaagagacgctggcgcaagatccccaagcgccctgg
ctccgtgcaccgcactccctcccgccaatgctgatagtcccctctcctgc
atccctccaccccagtccccgggaaaggccggtgggaagagggcaggggc
tgcatcctcagccccagggccctgctcaggcctcacctggcttcccagga
ccctgggtcaccttcctgggcagcccagagagaccctgccaactttccag
acttcgctattcccaggcagggagggaaacccggggaactggtttttctg
ttccctgctgggtgggaatgcgctcttcaccaggaagaaggcccgggagg
aggatccgggctttggactccttgtttgcctttaggcaggaagtcaggag
ccagcagggcgggccaggagaaagaaggcttaacattaagtattccttgg
cccagcagcggcccagattgcggtgtgagatggtgccccctggggggcac
agccagaaactgaactggccgctgggagaaaagccagatgacagggagct
ggggaatcccctcgcttcataggcagagcccgcccacctgggccctaggc
atactctccaggattgtccacaaatgtcctcagagggtccctaggtgggt
caactccaaggcaaatgtccaagcatcagcaagacaatgacactggaagg
gtccggcttggctagtcacatatcaagtcccgaggcagcaacaggaccag
gagccaggtgtcctgactgtcctacaatatcattttcctgggagtgggag
tcaagtgtgcctgctatccagccgcaaatccataccccctgccccagaga
agcctcagtccctccctcctggctcacagccaccacctggatggatctgc
tccatgcagatctagccaggcctcccgcatgctgcctgcctccggccctg
ccccacacaggcctggcccagccagcaggttctctcctgtgagctcccca
atccaacccatgcatggcctcccagccacccggatctccaggcccagcct
ggccccaaatgttctttcctttcatcctcagcaagtgctgagtctgtgaa
taaagccacataaccagcgggcaaaaaaaaaaaaaaaaaaaaaaaaaaaa
aaaaaaaaaaaaa
CHAT choline acetyltransferase
NM_020549 2485 bp mRNA linear PRI 09-DEC-2007
DEFINITION Homo sapiens choline acetyltransferase (CHAT), transcript
variant
                      M, mRNA.
ACCESSION NM_020549
VERSION NM_020549.3 GI: 119433674
KEYWORDS •
>/tmp/readseq.in.25404 [Unknown form], 2487 bases, 1C4A checksum.
tggggttggggaagtgcggtgactgggaaatgctgagctaggggcaggag
gcatgggcgggacagtgttctgtgcccccttctagagcctaaatttgttg
cccgagttcctccgggaagcgctccgggtagattctgggggccgggagct
gagatccctgggcggggagctggggaagggatggggctgaggacagcgaa
gaagagggggcttgggggaggggggaaatggaagagagaggagggaggag
gtacaagaggaaggagagaagtgcggccagcttgctttctccagtcgggt
ggccgcggggacccgggcgacgtcggaggccctgccgggaacccaggctg
cagcccccacccccgcgctgcgacacgccccccaccccttccggctcaca
cccccgcccacactcctgagtggtgcggtgcagcgtcggccgaggcagca
gagccgaggagagcaggtccacacctctgcatccctgcaccaggactcac
caagacgcccatcctggaaaaggtcccccgtaagatggcagcaaaaactc
ccagcagtgaggagtctgggctgcccaaactgcccgtgcccccgctgcag
cagaccctggccacgtacctgcagtgcatgcgacacttggtgtctgagga
gcagttcaggaagagccaggccattgtgcagcagtttggggcccctggtg
gcctcggcgagaccctgcagcagaaactcctggagcggcaggagaagaca
gccaactgggtgtctgagtactggctgaatgacatgtatctcaacaaccg
cctggccctgcctgtcaactccagccctgccgtgatctttgctcggcagc
acttccctggcaccgatgaccagctgaggtttgcagccagcctcatctct
ggtgtactcagctacaaggccctgctggacagccactccattcccactga
ctgtgccaaaggccagctgtcagggcagcccctttgcatgaagcaatact
atgggctcttctcctcctaccggctccccggccatacccaggacacgctg
gtggctcagaacagcagcatcatgccggagcctgagcacgtcatcgtagc
ctgctgcaatcagttctttgtcttggatgttgtcattaatttccgccgtc
tcagtgagggggatctgttcactcagttgagaaagatagtcaaaatggct
tccaacgaggacgagcgtttgcctccaattggcctgctgacgtctgacgg
gaggagcgagtgggccgaggccaggacggtcctcgtgaaagactccacca
accgggactcgctggacatgattgagcgctgcatctgccttgtatgcctg
gacgcgccaggaggcgtggagctcagcgacacccacagggcactccagct
ccttcacggcggaggctacagcaagaacggggccaatcgctggtacgaca
agtccctgcagtttgtggtgggccgagacggcacctgcggtgtggtgtgc
gaacactccccattcgatggcatcgtcctggtgcagtgcactgagcatct
gctcaagcacgtgacgcagagcagcaggaagctgatccgagcagactccg
tcagcgagctccccgccccccggaggctgcggtggaaatgctccccggaa
attcaaggccacttagcctcctcggcagaaaaacttcaacgaatagtaaa
gaaccttgacttcattgtctataagtttgacaactatgggaaaacattca
ttaagaagcagaaatgcagccctgatgccttcatccaggtggccctccag
ctggccttctacaggctccatcgaagactggtgcccacctacgagagcgc
gtccatccgccgattccaggagggacgcgtggacaacatcagatcggcca
ctccagaggcactggcttttgtgagagccgtgactgaccacaaggctgct
gtgccagcttctgagaagcttctgctcctgaaggatgccatccgtgccca
gactgcatacacagtcatggccataacagggatggccattgacaaccacc
tgctggcactgcgggagctggcccgggccatgtgcaaggagctgcccgag
atgttcatggatgaaacctacctgatgagcaaccggtttgtcctctccac
tagccaggtgcccacaaccacggagatgttctgctgctatggtcctgtgg
tcccaaatgggtatggtgcctgctacaacccccagccagagaccatcctt
ttctgcatctctagctttcacagctgcaaagagacttcttctagcaagtt
tgcaaaagctgtggaagaaagcctcattgacatgagagacctctgcagtc
tgctgccgcctactgagagcaagccattggcaacaaaggaaaaagccacg
aggcccagccagggacaccaaccttgactcctgccactaggtttcacctc
ccaaacccagcctctagaacagccagaccctgcag//
NRG1: neuregulin 1: Neuregulin 1 (NRG1)
HUMGGFB 1199 bp mRNA linear PRI 12-JUN-1993
DEFINITION Human recombinant glial growth factor mRNA, complete cds
and
                      flanking region.
ACCESSION L12261
VERSION L12261.1 GI: 292049
KEYWORDS glial growth factor; neuregulin.
SOURCE Homo sapiens (human)
>/tmp/readseq.in.25408 [Unknown form], 1199 bases, 209A checksum.
gcgcggaggccaggagctgagcggcggcggctgccggacgatgggagcgt
gagcaggacggtgataacctctccccgatcgggttgcgagggcgccgggc
agaggccaggacgcgagccgccagcggcgggacccatcgacgacttcccg
gggcgacaggagcagccccgagagccagggcgagcgcccgttccaggtgg
ccggaccgcccgccgcgtccgcgccgcgctccctgcaggcaacgggagac
gcccccgcgcagcgcgagcgcctcagcgcggccgctcgctctccccatcg
agggacaaacttttcccaaacccgatccgagcccttggaccaaactcgcc
tgcgccgagagccgtccgcgtagagcgctccgtctccggcgagatgtccg
agcgcaaagaaggcagaggcaaagggaagggcaagaagaaggagcgaggc
tccggcaagaagccggagtccgcggcgggcagccagagcccagccttgcc
tccccgattgaaagagatgaaaagccaggaatcggctgcaggttccaaac
tagtccttcggtgtgaaaccagttctgaatactcctctctcagattcaag
tggttcaagaatgggaatgaattgaatcgaaaaaacaaaccacaaaatat
caagatacaaaaaaagccagggaagtcagaacttcgcattaacaaagcat
cactggctgattctggagagtatatgtgcaaagtgatcagcaaattagga
aatgacagtgcctctgccaatatcaccatcgtggaatcaaacgagatcat
cactggtatgccagcctcaactgaaggagcatatgtgtcttcagagtctc
ccattagaatatcagtatccacagaaggagcaaatacttcttcatctaca
tctacatccaccactgggacaagccatcttgtaaaatgtgcggagaagga
gaaaactttctgtgtgaatggaggggagtgcttcatggtgaaagaccttt
caaacccctcgagatacttgtgcaagtgcccaaatgagtttactggtgat
cgctgccaaaactacgtaatggccagcttctacagtacgtccactccctt
tctgtctctgcctgaataggagcatgctcagttggtgctgctttcttgtt
gctgcatctcccctcagattccacctagagctagatgtgtcttaccaga
```

All publications mentioned in the present specification, and references cited in said publications, are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1-12. (canceled)

13. A method of determining the likelihood of a general response to risperidone treatment in a patient comprising detecting the genotypes of one or more polymorphisms in one or more genes in a sample of said patient, wherein said genes are selected from the group consisting of ADRA1A, DRD2 and DRD4.

14. (canceled)

15. The method of claim 13, further comprising detecting the genotypes of one or more polymorphisms in one or more genes selected from the group consisting of 5 HT1A, CYP2D6*4, and 5-HT2A.

16. (canceled)

17. The method of claim 13, wherein said one or more polymorphisms in ADRA1A comprises Arg492/Cys492, wherein said one or more polymorphisms in DRD2 comprises Tag I A1/A2, and wherein said one or more polymorphisms in DRD4 comprises −521 C/T.

18. The method of claim 15, wherein said one or more polymorphisms in 5-HT1A comprises −1018 C/G, wherein said one or more polymorphisms in CYP2D6 comprises *4 A/G, and wherein said one or more polymorphisms in 5-HT2A comprises 102 T/C.

19-20. (canceled)

21. The method of claim 17, wherein said likelihood of a general response to risperidone treatment in said patient (LoR) is calculated according to the following algorithm: 1−(−1.565+2.293A1−0.821A2+1.521B1−0.421C1+1.443C2)], wherein A1=ADRA1A Arg492/Arg492, A2=ADRA1A Arg492/Cys492, B1=DRD2 Taq I A2/A2, C1=DRD4−521 C/C and C2=DRD4−521 C/T.

22. The method of claim 18, wherein said likelihood of a general response to risperidone treatment in said patient (LoR) is calculated according to the following algorithm: =[1−(−5.381+2.831A1−0.542A2+1.904B1−0.310C1+2.160C2+22.479D1+1.68D2−19.014E1+0.424E2+1.347F1+2.166F2)], wherein A1=ADRA1A Arg492/Arg492, A2=ADRA1A Arg492/Cys492, B1=DRD2 Taq I A2/A2, C1=DRD4−521 C/C and C2=DRD4−521 C/T, D1=5-HT1A−1018 C/C, D2=5-HT1A−1018 C/G, E1=CYP2D6*4 A/A, E2=CYP2D6*4 A/G, F1=5-HT2A 102 T/T, and F2=5-HT2A 102 T/C.

23. The method of claim 21, wherein said response is measured by PANSS, and said response is a therapeutically effective response comprising at least a 30% decrease in PANSS.

24. The method of claim 17, wherein said likelihood of a general response to risperidone treatment in said patient (LoR) is calculated according to the following algorithm: LoR=[1−(−0.615−0.723A1−0.917A2+0.890B1−0.961C1+1.057C2)] wherein A1=ADRA1 A Arg492/Arg492, A2=ADRA1 A Arg492/Cys492, B1=DRD2 Taq I A2/A2, C1=DRD4−521 C/C and C2=DRD4−521 C/T.

25. The method of claim 18, wherein said likelihood of a general response to risperidone treatment in said patient (LoR) is calculated according to the following algorithm: =LoR=[1−(−0.185−1.07A1−1.494A2+0.798B1−0.301C1+0.81C2+1.982D1+0.527D2−21.389E1+0.409E2−2.566F1−0.627F2)], wherein A1=ADRA1A Arg492/Arg492, A2=ADRA1A Arg492/Cys492, B1=DRD2 Taq I A2/A2, C1=DRD4−521 C/C and C2=DRD4−521 C/T, D1=5-HT1 A−1018 C/C, D2=5-HT1A−1018 C/G, E1=CYP2D6*4 A/A, E2=CYP2D6*4 A/G, F1=5-HT2A 102 T/T, and F2=5-HT2A 102 T/C.

26. The method of claim 24, wherein said response is measured by GAF, and said response is a therapeutically effective response comprising an improvement of 20 points or more in GAF scale.

27-50. (canceled)

51. A kit for determining a genotype of an individual, wherein said kit comprises oligonucleotides for detection of genotypes of each polymorphism in the group consisting of: ADRA1A Arg492/Arg492, ADRA1A Arg492/Cys492, DRD2 Taq I A2/A2, DRD4−521 C/C and DRD4−521 C/T, 5-HT1A−1018 C/C, 5-HT1 A−1018 C/G, CYP2D6*4 A/A, CYP2D6*4 A/G, 5-HT2A 102 T/T, and 5-HT2A 102 T/C.

52. The kit of claim 51, wherein said oligonucleotides comprise oligonucleotides with sequences selected from the group consisting of: SEQ ID NO:3, SEQ ID NO:4, SEQ ID NO:20, SEQ ID NO:21, SEQ ID NO:24, SEQ ID NO:25, SEQ ID NO:26, SEQ ID NO: 27, SEQ ID NO:18, SEQ ID NO:19, SEQ ID NO:28 and SEQ ID NO:29.

53-58. (canceled)

59. An isolated nucleic acid comprising a polymorphism selected from the group consisting of: ADRA1A Arg492Cys ADRA1A−6274 T/C ChAT rs1880676 G/A, COMTVal102/158Met, CYP2D6*4 EM/PM, DRD2 Taq IA A1/A2, DRD4−521 C/T, 5-HT1A−1018 G/C, 5-HT2A 102 T/C, 5-HT2A−1438 G/A, 5-HT2C rs475717 A/C, 5-HT2C Cys23Ser, 5-HTT 2630 C/T, M1−12,064 T/C, NRG1 SNP8NRG221533.

60. The method of claim 22, wherein said response is measured by PANSS, and said response is a therapeutically effective response comprising at least a 30% decrease in PANSS.

61. The method of claim 25, wherein said response is measured by GAF, and said response is a therapeutically effective response comprising an improvement of 20 points or more in GAF scale.


SEQUENCE LISTING (76 sequences)

SEQ ID NO: 1 - DNA, 1269 bp, Homo sapiens
atggatgtgctcagccctggtcagggcaacaacaccacatcaccaccggctccctttgagaccggcggcaacactactggtatctccgacgtgaccgtcagctaccaagtgatcacctctctgctgctgggcacgctcatcttctgcgcggtgctgggcaatgcgtgcgtggtggctgccatcgccttggagcgctccctgcagaacgtggccaattatcttattggctctttggcggtcaccgacctcatggtgtcggtgttggtgctgcccatggccgcgctgtatcaggtgctcaacaagtggacactgggccaggtaacctgcgacctgttcatcgccctcgacgtgctgtgctgcacctcatccatcttgcacctgtgcgccatcgcgctggacaggtactgggccatcacggaccccatcgactacgtgaacaagaggacgccccggcgcgccgctgcgctcatctcgctcacttggcttattggcttcctcatctctatcccgcccatgctgggctggcgcaccccggaagaccgctcggaccccgacgcatgcaccattagcaaggatcatggctacactatctattccacctttggagctttctacatcccgctgctgctcatgctggttctctatgggcgcatattccgagctgcgcgcttccgcatccgcaagacggtcaaaaaggtggagaagaccggagcggacacccgccatggagcatctcccgccccgcagcccaagaagagtgtgaatggagagtcggggagcaggaactggaggctgggcgtggagagcaaggctgggggtgctctgtgcgccaatggcgcggtgaggcaaggtgacgatggcgccgccctggaggtgatcgaggtgcaccgagtgggcaactccaaagagcacttgcctctgcccagcgaggctggtcctaccccttgtgcccccgcctctttcgagaggaaaaatgagcgcaacgccgaggcgaagcgcaagatggccctggcccgagagaggaagacagtgaagacgctgggcatcatcatgggcaccttcatcctctgctggctgcccttcttcatcgtggctcttgttctgcccttctgcgagagcagctgccacatgcccaccctgttgggcgccataatcaattggctgggctactccaactctctgcttaaccccgtcatttacgcatacttcaacaaggactttcaaaacgcgtttaagaagatcattaagtgtaagttctgccgccagtga

SEQ ID NO: 2 - DNA, 3009 bp, Homo sapiens
atccagccccgggagaacagcatgtacaccagcctcagtgttacagagtgtgggtacatcaaggtgaatggtgagcagaaactataacctgttagtccttctacacctcatctgctacaagttctggcttagacatggatattctttgtgaagaaaatacttctttgagctcaactacgaactccctaatgcaattaaatgatgacaccaggctctacagtaatgactttaactccggagaagctaacacttctgatgcatttaactggacagtcgactctgaaaatcgaaccaacctttcctgtgaagggtgcctctcaccgtcgtgtctctccttacttcatctccaggaaaaaaactggtctgctttactgacagccgtagtgattattctaactattgctggaaacatactcgtcatcatggcagtgtccctagagaaaaagctgcagaatgccaccaactatttcctgatgtcacttgccatagctgatatgctgctgggtttccttgtcatgcccgtgtccatgttaaccatcctgtatgggtaccggtggcctctgccgagcaagctttgtgcagtctggatttacctggacgtgctcttctccacggcctccatcatgcacctctgcgccatctcgctggaccgctacgtcgccatccagaatcccatccaccacagccgcttcaactccagaactaaggcatttctgaaaatcattgctgtttggaccatatcagtaggtatatccatgccaataccagtctttgggctacaggacgattcgaaggtctttaaggaggggagttgcttactcgccgatgataactttgtcctgatcggctcttttgtgtcatttttcattcccttaaccatcatggtgatcacctactttctaactatcaagtcactccagaaagaagctactttgtgtgtaagtgatcttggcacacgggccaaattagcttctttcagcttcctccctcagagttctttgtcttcagaaaagctcttccagcggtcgatccatagggagccagggtcctacacaggcaggaggactatgcagtccatcagcaatgagcaaaaggcatgcaaggtgctgggcatcgtcttcttcctgtttgtggtgatgtggtgccctttcttcatcacaaacatcatggccgtcatctgcaaagagtcctgcaatgaggatgtcattggggccctgctcaatgtgtttgtttggatcggttatctctcttcagcagtcaacccactagtctacacactgttcaacaagacctataggtcagccttttcacggtatattcagtgtcagtacaaggaaaacaaaaaaccattgcagttaattttagtgaacacaataccggctttggcctacaagtctagccaacttcaaatgggacaaaaaaagaattcaaagcaagatgccaagacaacagataatgactgctcaatggttgctctaggaaagcagcattctgaagaggcttctaaagacaatagcgacggagtgaatgaaaaggtgagctgtgtgtgataggctagttgccgtggcaactgtggaaggcacactgagcaagttttcacctatctggaaaaaaaaaaatatgagattggaaaaaattagacaagtctagtggaaccaacgatcatatctgtatgcctcattttattctgtcaatgaaaagcggggttcaatgctacaaaatgtgtgcttggaaaatgttctgacagcatttcagctgtgagctttctgatacttatttataacattgtaaatgatatgtctttaaaatgattcacttttattgtataattatgaagccctaagtaaatctaaattaacttctattttcaagtggaaaccttgctgctatgctgttcattgatgacatgggattgagttggttacctattgctgtaaataaaaatagctataaatagtgaaaattttattgaatataatggcctcttaaaaattatctttaaaacttactatggtatatattttgaaaggagaaaaaaaaagccactaaggtcagtgttataaaatctgtattgctaagataattaaatgaaatacttgacaacatttttcattcctgctttttcatagataccattttgaaatattcacaaggttgctggcatttgctgcatttcaagttaattctcagaagtgaaaaagacttcaaatgttattcaataactattgctgctttctcttctacttcttgtgctttactctgaatttccagtgtggtcttgtttaatatttgttcctctaggtaaactagcaaaaggatgatttaacattaccaaatgcctttctagcaattgcttctctaaaacagcactatcgaggtatttggtaacttgctgtgaaatgactgcatcatgcatgcactcttttgagcagtaaatgtatattgatgtaactgtgtcaggattgaggatgaactcaggtttccggctactgacagtggtagagtcctaggacatctctgtaaaaagcaggtgactttcctatgacactcatcaggtaaactgatgctttcagatccatcggtttatactatttattaaaaccattctgcttggttccacaatcatctattgagtgtacatttatgtgtgaagcaaatttctagatatgagaaatataaaaataattaaaacaaaatccttgccttcaaacgaaatggctcggccaggcacggaggctcgtgcatgtaatcctagcactttgggaggctgagatgggaggatcacttgaggccaagagtttgagaccaacctgggtaacaaagtgagacctccctgtctctacaaaaaaaatcaaaaaattatctgatccttgtggcacacaactgtggtcccagctacaggggaggctgagacgcaaggatcacttgagcccagaagctcaaggctgcagtgagccaagttcacaccactgccatttcctcctgggcaacagagtgagaccctatcac

SEQ ID NO: 3 - DNA, 20 bp, Artificial Sequence
  Description of Artificial Sequence: Synthetic oligonucleotide
atgctccagccaagagttca

SEQ ID NO: 4 - DNA, 20 bp, Artificial Sequence
  Description of Artificial Sequence: Synthetic oligonucleotide
tccaagaagagctggccttc

SEQ ID NO: 5 - DNA, 24 bp, Artificial Sequence
  Description of Artificial Sequence: Synthetic oligonucleotide
tatgtatatgctattgtctgaaag

SEQ ID NO: 6 - DNA, 20 bp, Artificial Sequence
  Description of Artificial Sequence: Synthetic oligonucleotide
aagcgcccattcttcataga

SEQ ID NO: 7 - DNA, 4775 bp, Homo sapiens
acccgcgcgaggtaggcgctctggtgcttgcggaggacgcttccttcctcagatgcaccgatcttcccgatactgcctttggagcggctagattgctagccttggctgctccattggcctgccttgccccttacctgccgattgcatatgaactcttcttctgtctgtacatcgttgtcgtcggagtcgtcgcgatcgtcgtggcgctcgtgtgatggccttcgtccgtttagagtagtgtagttagttaggggccaacgaagaagaaagaagacgcgattagtgcagagatgctggaggtggtcagttactaagctagagtaagatagcggagcgaaaagagccaaacctagccggggggcgcacggtcacccaaaggaggtcgactcgccggcgcttcctatcgcgccgagctccctccattcctctccctccgccgaggcgcgaggttgcggcgcgcagcgcagcgcagctcagcgcaccgactgccgcgggctccgctgggcgattgcagccgagtccgtttctcgtctagctgccgccgcggcgaccgctgcctggtcttcctcccggacgctagtgggttatcagctaacacccgcgagcatctataacataggccaactgacgccatccttcaaaaacaactaaaggatgatatgatgaacctagcctgttaatttcgtcttctcaattttaaactttggttgcttaagactgaagcaatcatggtgaacctgaggaatgcggtgcattcattccttgtgcacctaattggcctattggtttggcaatgtgatatttctgtgagcccagtagcagctatagtaactgacattttcaatacctccgatggtggacgcttcaaattcccagacggggtacaaaactggccagcactttcaatcgtcatcataataatcatgacaataggtggcaacatccttgtgatcatggcagtaagcatggaaaagaaactgcacaatgccaccaattacttcttaatgtccctagccattgctgatatgctagtgggactacttgtcatgcccctgtctctcctggcaatcctttatgattatgtctggccactacctagatatttgtgccccgtctggatttctttagatgttttattttcaacagcgtccatcatgcacctctgcgctatatcgctggatcggtatgtagcaatacgtaatcctattgagcatagccgtttcaattcgcggactaaggccatcatgaagattgctattgtttgggcaatttctataggtgtatcagttcctatccctgtgattggactgagggacgaagaaaaggtgttcgtgaacaacacgacgtgcgtgctcaacgacccaaatttcgttcttattgggtccttcgtagctttcttcataccgctgacgattatggtgattacgtattgcctgaccatctacgttctgcgccgacaagctttgatgttactgcacggccacaccgaggaaccgcctggactaagtctggatttcctgaagtgctgcaagaggaatacggccgaggaagagaactctgcaaaccctaaccaagaccagaacgcacgccgaagaaagaagaaggagagacgtcctaggggcaccatgcaggctatcaacaatgaaagaaaagcttcgaaagtccttgggattgttttctttgtgtttctgatcatgtggtgcccatttttcattaccaatattctgtctgttctttgtgagaagtcctgtaaccaaaagctcatggaaaagcttctgaatgtgtttgtttggattggctatgtttgttcaggaatcaatcctctggtgtatactctgttcaacaaaatttaccgaagggcattctccaactatttgcgttgcaattataaggtagagaaaaagcctcctgtcaggcagattccaagagttgccgccactgctttgtctgggagggagcttaatgttaacatttatcggcataccaatgaaccggtgatcgagaaagccagtgacaatgagcccggtatagagatgcaagttgagaatttagagttaccagtaaatccctccagtgtggttagcgaaaggattagcagtgtgtgagaaagaacagcacagtcttttcctacggtacaagctacatatgtaggaaaattttcttctttaatttttctgttggtcttaactaatgtaaatattgctgtctgaaaaagtgtttttacatatagctttgcaaccttgtactttacaatcatgcctacattagtgagatttagggttctatatttactgtttataataggtggagactaacttattttgattgtttgatgaataaaatgtttatttttgctctccctcccttctttccttccttttttcctttcttccttcctttctctctttcttttgtgcatatggcaacgttcatgttcatctcaggtggcatttgcaggtgaccagaatgaggcacatgacagtggttatatttcaaccacacctaaattaacaaattcagtggacatttgttctgggttaacagtaaatatacactttacattcttgctctgctcatctacacatataaacacagtaagataggttctgctttctgatacatctgtcagtgagtcagaggcagaacctagtcttgttgttcatataggggcaaaaatttgacattgtcagaatgttgtgttggtatttactgcaatgtctgtccctaaacatagtggtattttaacatagcagctggttaaccgggactacagaagtggaaggataatgagatgtaatacaccaaatagcttttcacttcttaaggacagtgttcaaattctgattattacaacaagcaaactgaaattagtgttttcattctggtccttagtaaattcctaattctatgattaaactgggaaatgagatcccagagttatttcccaacccaggattcaacatcaattgggttttgatctcagcatcctggaaatttgtgtgcttcacacaaagtgaaattagtattttgagccttattaaaatattttcttaattatggtacctctgtctataggacttaatttagcagtccatttttgagtaaaacttgtattggaagtatagatggtagaaactttggaagttttacttgattaaggactacagaattgggcccttagaatgtgaaaaaaaaaagtaattaaaaagacacttttaccgaactcgggattacagaaacacggagtttccatttggattttaaacaaaatttatgtcattttcagatccttccaaactctctagtgcaggaaaaggctgcagctaatttgtgaaagtggcaagctcttcattgcactgcagttatttaccagaagtttaaatctttgttaaaatatagtgttgtgttacaataagtgttggccatcatttcattcgtgggcctgctgctctctaagaattcagtagcattttaatagtttctaaaccatgaaaagttttcaagcattgctaaagtcaggccattcagtctatgctgtgtgcagagtatacaagtgtttctagtaacagtatttccatacgtgcccatttcacacaactgtggataaattttggaagaattcatgatgctagttcttacgcttgacagttacttacacacctgagaatgtgcctctcagtatcttaaaattggttaatgaaaaatctgaatttctaaaacccttggtctgtgttctcaacacacagtatagataaatccaatagtctgccacaagggcagtggaagagctgctgtatttgaggaaactcatacagtctctatttgatttgcaacactggccaaacatcagtcatttgcttgagcatgcccaaatattacatgaaagtcaagtctacctgccttgcctgttaggtctgttgaagtgcatgttaaaataattatatgaagcagaatgagatgatttaattcttaccgaaatgaaaatggctgaagaaacacagcatgcatttagcatgagttctgcacatacagatggtgtcctgcatgtatgccatgtatgttgcatgaatccatcgatttgtattaatgtagggcagaatagctgatagaagaaggactgaagaaaatccttcagcaatccttaaaaagaccatgcattcagatctgaagtagtgtgagtgttagaaaaaactggaaacatctgatttctgaactatcagggcaagctcatagcacatgttttacaaagaaacaaaatataaatcacagatttccaaaagtactagcaataagttgaatgataatagctcacagcacatttgttaatgattcttgtgtcatcaagtagtagtacttaatagtacccaacctggtaattatcctcaagttgtgtgctattcgtaagttctgtgcagtttggtatgaaacaaatatactcatttggatataaatcttacccttcaatgttaaatctacaaacttttataaatgttttaaagaagtccatgtgataattgtaaaggtgatgaatttaccatcaaacaaatcattttgatgtattattatatatgtatatctgtgtaagacacgtgcaacagactgccttatattattttctgtaattcttctcctttgtcaaatggtattttttgtgaatggttgcaaagtgttgtcttattcctaattcctgtatgttatccactacaggttttatgagacttcctattaatttattaaatttattaaatgttgaaaaaaaaaaaaaaaaaaaaaaa

SEQ ID NO: 8 - DNA, 2775 bp, Homo sapiens
acagccagcgccgccgggtgcctcgagggcgcgaggccagcccgcctgcccagcccgggaccagcctccccgcgcagcctggcaggtctcctggaggcaaggcgaccttgcttgccctctcttgcagaataacaaggggcttagccacaggagttgctggcaagtggaaagaagaacaaatgagtcaatcccgacgtgtcaatcccgacgatagagagctcggaggtgatccacaaatccaagcacccagagatcaattgggatccttggcagatggacatcagtgtcatttactaaccagcaggatggagacgacgcccttgaattctcagaagcagctatcagcgtgtgaagatggagaagattgtcaggaaaacggagttctacagaaggttgttcccaccccaggggacaaagtggagtccgggcaaatatccaatgggtactcagcagttccaagtcctggtgcgggagatgacacacggcactctatcccagcgaccaccaccaccctagtggctgagcttcatcaaggggaacgggagacctggggcaagaaggtggatttccttctctcagtgattggctatgctgtggacctgggcaatgtctggcgcttcccctacatatgttaccagaatggagggggggcattcctcctcccctacaccatcatggccatttttgggggaatcccgctcttttacatggagctcgcactgggacagtaccaccgaaatggatgcatttcaatatggaggaaaatctgcccgattttcaaagggattggttatgccatctgcatcattgccttttacattgcttcctactacaacaccatcatggcctgggcgctatactacctcatctcctccttcacggaccagctgccctggaccagctgcaagaactcctggaacactggcaactgcaccaattacttctccgaggacaacatcacctggaccctccattccacgtcccctgctgaagaattttacacgcgccacgtcctgcagatccaccggtctaaggggctccaggacctggggggcatcagctggcagctggccctctgcatcatgctgatcttcactgttatctacttcagcatctggaaaggcgtcaagacctctggcaaggtggtgtgggtgacagccaccttcccttatatcatcctttctgtcctgctggtgaggggtgccaccctccctggagcctggaggggtgttctcttctacttgaaacccaattggcagaaactcctggagacaggggtgtggatagatgcagccgctcagatcttcttctctcttggtccgggctttggggtcctgctggcttttgctagctacaacaagttcaacaacaactgctaccaagatgccctggtgaccagcgtggtgaactgcatgacgagcttcgtttcgggatttgtcatcttcacagtgctcggttacatggctgagatgaggaatgaagatgtgtctgaggtggccaaagacgcaggtcccagcctcctcttcatcacgtatgcagaagcgatagccaacatgccagcgtccactttctttgccatcatcttctttctgatgttaatcacgctgggcttggacagcacgtttgcaggcttggagggggtgatcacggctgtgctggatgagttcccacacgtctgggccaagcgccgggagcggttcgtgctcgccgtggtcatcacctgcttctttggatccctggtcaccctgacttttggaggggcctacgtggtgaagctgctggaggagtatgccacggggcccgcagtgctcactgtcgcgctgatcgaagcagtcgctgtgtcttggttctatggcatcactcagttctgcagggacgtgaaggaaatgctcggcttcagcccggggtggttctggaggatctgctgggtggccatcagccctctgtttctcctgttcatcatttgcagttttctgatgagcccgccacaactacgacttttccaatataattatccttactggagtatcatcttgggttactgcataggaacctcatctttcatttgcatccccacatatatagcttatcggttgatcatcactccagggacatttaaagagcgtattattaaaagtattaccccagaaacaccaacagaaattccttgtggggacatccgcttgaatgctgtgtaacacactcaccgagaggaaaaaggcttctccacaacctcctcctccagttctgatgaggcacgcctgccttctcccctccaagtgaatgagtttccagctaagcctgatgatggaagggccttctccacagggacacagtctggtgcccagactcaaggcctccagccacttatttccatggattcccctggacatattcccatggtagactgtgacacagctgagctggcctattttggacgtgtgaggatgtggatggaggtgatgaaaaccaccctatcatcagttaggattaggtttagaatcaagtctgtgaaagtctcctgtatcatttcttggtatgatcattggtatctgatatctgtttgcttctaaaggtttcactgttcatgaatacgtaaactgcgtaggagagaacagggatgctatctcgctagccatatattttctgagtagcatatataattttattgctggaatctactagaaccttctaatccatgtgctgctgtggcatcaggaaaggaagatgtaagaagctaaaatgaaaaatagtgtgtccatgcaaaaaaaaaaa

SEQ ID NO: 9 - DNA, 2304 bp, Homo sapiens
gaattccgaatcatgtgcagaatgctgaatcttcccccagccaggacgaataagacagcgcggaaaagcagattctcgtaattctggaattgcatgttgcaaggagtctcctggatcttcgcacccagcttcgggtagggagggagtccgggtcccgggctaggccagcccggcaggtggagagggtccccggcagccccgcgcgcccctggccatgtctttaatgccctgccccttcatgtggccttctgagggttcccagggctggccagggttgtttcccacccgcgcgcgcgctctcacccccagccaaacccacctggcagggctccctccagccgagaccttttgattcccggctcccgcgctcccgcctccgcgccagcccgggaggtggccctggacagccggacctcgcccggccccggctgggaccatggtgtttctctcgggaaatgcttccgacagctccaactgcacccaaccgccggcaccggtgaacatttccaaggccattctgctcggggtgatcttggggggcctcattcttttcggggtgctgggtaacatcctagtgatcctctccgtagcctgtcaccgacacctgcactcagtcacgcactactacatcgtcaacctggcggtggccgacctcctgctcacctccacggtgctgcccttctccgccatcttcgaggtcctaggctactgggccttcggcagggtcttctgcaacatctgggcggcagtggatgtgctgtgctgcaccgcgtccatcatgggcctctgcatcatctccatcgaccgctacatcggcgtgagctacccgctgcgctacccaaccatcgtcacccagaggaggggtctcatggctctgctctgcgtctgggcactctccctggtcatatccattggacccctgttcggctggaggcagccggcccccgaggacgagaccatctgccagatcaacgaggagccgggctacgtgctcttctcagcgctgggctccttctacctgcctctggccatcatcctggtcatgtactgccgcgtctacgtggtggccaagagggagagccggggcctcaagtctggcctcaagaccgacaagtcggactcggagcaagtgacgctccgcatccatcggaaaaacgccccggcaggaggcagcgggatggccagcgccaagaccaagacgcacttctcagtgaggctcctcaagttctcccgggagaagaaagcggccaaaacgctgggcatcgtggtcggctgcttcgtcctctgctggctgccttttttcttagtcatgcccattgggtctttcttccctgatttcaagccctctgaaacagtttttaaaatagtattttggctcggatatctaaacagctgcatcaaccccatcatatacccatgctccagccaagagttcaaaaaggcctttcagaatgtcttgagaatccagtgtctctgcagaaagcagtcttccaaacatgccctgggctacaccctgcacccgcccagccaggccgtggaagggcaacacaaggacatggtgcgcatccccgtgggatcaagagagaccttctacaggatctccaagacggatggcgtttgtgaatggaaatttttctcttccatgccccgtggatctgccaggattacagtgtccaaagaccaatcctcctgtaccacagcccggacgaagtctcgctctgtcaccaggctggagtgcagtggcatgatcttggctcactgcaacctccgcctcccgggttcaagagattctcctgcctcagcctcccaagcagctgggactacagggatgtgccaccaggccgacgccaccaggcccagctaatttttgtatttttagtagagacggggtttcaccatgttggccaggatgatctcgatctcttgacctcatgatctgcctgcctcagcctcccaaagtgctgggattacaggcgtgagccaccgtgcccggcccaactattttttttttttatcttttttaacagtgcaatcctttctgtggatgaaatcttgctcagaagctcaatatgcaaaagaaagaaaaacagcagggctggacggatgttgggagtggggtaagaccccaaccactcagaaccacccccccaacacacacacacattctctccatggtgactggtgaggggcctctagagggtacatagtacaccatggagcacggtttaagcaccactggactacacattcttctgtggcagttatcttaccttcccatagacacccagcccatagccattggtt

SEQ ID NO: 10 - DNA, 1567 bp, Homo sapiens
atggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtatgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctcctagcccagaggctctaatgtacaataaagcaatgtggtagttcc

SEQ ID NO: 11 - DNA, 21 bp, Artificial Sequence
  Description of Artificial Sequence: Synthetic oligonucleotide
agatgtggccggaatgcagag

SEQ ID NO: 12 - DNA, 22 bp, Artificial Sequence
  Description of Artificial Sequence: Synthetic oligonucleotide
gagatgtggccggaatgcagaa

SEQ ID NO: 13 - DNA, 26 bp, Artificial Sequence
  Description of Artificial Sequence: Synthetic oligonucleotide
catacctactgtgctcagtgcttcat

SEQ ID NO: 14 - DNA, 2643 bp, Homo sapiens
ggcagccgtccggggccgccactctcctcggccggtccctggctcccggaggcggccgcgcgtggatgcggcgggagctggaagcctcaagcagccggcgccgtctctgccccggggcgccctatggcttgaagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacccggacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccggccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccaggcaacttcagtcctgggagacccatgtaaataccagactgcaggttggaccccagagattcccaagccaaaaaccttagctccctcccgcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtaaaaaaaaaaaaaaa

SEQ ID NO: 15 - DNA, 1360 bp, Homo sapiens
atggggaaccgcagcaccgcggacgcggacgggctgctggctgggcgcgggccggccgcgggggcatctgcgggggcatctgcggggctggctgggcagggcgcggcggcgctggtggggggcgtgctgctcatcggcgcggtgctcgcggggaactcgctcgtgtgcgtgagcgtggccaccgagcgcgccctgcagacgcccaccaactccttcatcgtgagcctggcggccgccgacctcctcctcgctctcctggtgctgccgctcttcgtctactccgaggtccagggtggcgcgtggctgctgagcccccgcctgtgcgacgccctcatggccatggacgtcatgctgtgcaccgcctccatcttcaacctgtgcgccatcagcgtggacaggttcgtggccgtggccgtgccgctgcgctacaaccggcagggtgggagccgccggcagctgctgctcatcggcgccacgtggctgctgtccgcggcggtggcggcgcccgtactgtgcggcctcaacgacgtgcgcggccgcgaccccgccgtgtgccgcctggaggaccgcgactacgtggtctactcgtccgtgtgctccttcttcctaccctgcccgctcatgctgctgctctactgggccacgttccgcggcctgcagcgctgggaggtggcacgtcgcgccaagctgcacggccgcgcgccccgccgacccagcggccctggcccgccttcccccacgccacccgcgccccgcctcccccaggacccctgcggccccgactgtgcgccccccgcgcccggccttccccggggtccctgcggccccgactgtgcgcccgccgcgcccagcctcccccaggacccctgtggccccgactgtgcgccccccgcgcccggcctccccccggacccctgcggctccaactgtgctccccccgacgccgtcagagccgccgcgctcccaccccagactccaccgcagacccgcaggaggcggcgtgccaagatcaccggccgggagcgcaaggccatgagggtcctgccggtggtggtcggggccttcctgctgtgctggacgcccttcttcgtggtgcacatcacgcaggcgctgtgtcctgcctgctccgtgcccccgcggctggtcagcgccgtcacctggctgggctacgtcaacagcgccctcaaccccgtcatctacactgtcttcaacgccgagttccgcaacgtcttccgcaaggccctgcgtgcctgctgctgagccgggcacccccggacgccccccggcctgatggccaggcctcagggaccaaggagatggggagggcgcttttgtacgttaattaaacaaattccttccc

SEQ ID NO: 16 - DNA, 21 bp, Artificial Sequence
  Description of Artificial Sequence: Synthetic oligonucleotide
tcgtggacgccgtgattcagg

SEQ ID NO: 17 - DNA, 21 bp, Artificial Sequence
  Description of Artificial Sequence: Synthetic oligonucleotide
aggtctgacaacgggtcaggc

SEQ ID NO: 18 - DNA, 19 bp, Artificial Sequence
  Description of Artificial Sequence: Synthetic oligonucleotide
gccttcgccaaccactccg

SEQ ID NO: 19 - DNA, 20 bp, Artificial Sequence
  Description of Artificial Sequence: Synthetic oligonucleotide
aaatcctgctcttccgaggc

SEQ ID NO: 20 - DNA, 26 bp, Artificial Sequence
  Description of Artificial Sequence: Synthetic oligonucleotide
ccgtcgacggctggccaagttgtcta

SEQ ID NO: 21 - DNA, 26 bp, Artificial Sequence
  Description of Artificial Sequence: Synthetic oligonucleotide
ccgtcgacccttcctgagtgtcatca

SEQ ID NO: 22 - DNA, 1067 bp, Homo sapiens
gctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctggtactgaaggtgccagacgtgctcctgctgaccttctgcggctccgggctgtgtcctaaatgcaaagcacacctcggccgaggcctgcgccctgacatgctaacctctctgaactgcaacactggattgttcttttttaagactcaatcatgacttctttactaacactggctagctatattatcttatatactaatatcatgttttaaaaatataaaatagaaattaa

SEQ ID NO: 23 - DNA, 2863 bp, Homo sapiens
tggggctcaaattgggtgccctggtgaaggaggggggcacactccagaacctagtccaaccccagacgctgcctgaggcttccctccagctcccctcccttccttttctccctttcctccctccctctctttccctttctccctccccgctaaggctggcgtgccagggggtgggacatgccaatcactggctgtgcctctcccgctgccagcacagggcgcagctccccctgggagccaggtgtttgggtccctggagacgccgcaggcccccagggaggcagtggggctgaggaccctacagacccctcttcagccccgtggtgatgactttcccctgaggaagccctgtagcgtgcctggaggaaggggctctccaaccccagccccacctagccaccatgaacacttcagccccacctgctgtcagccccaacatcaccgtcctggcaccaggaaagggtccctggcaagtggccttcattgggatcaccacgggcctcctgtcgctagccacagtgacaggcaacctgctggtactcatctctttcaaggtcaacacggagctcaagacagtcaataactacttcctgctgagcctggcctgtgctgacctcatcatcggtaccttctccatgaacctctataccacgtacctgctcatgggccactgggctctgggcacgctggcttgtgacctctggctggccctggactatgtggccagcaatgcctccgtcatgaatctgctgctcatcagctttgaccgctacttctccgtgactcggcccctgagctaccgtgccaagcgcacaccccgccgggcagctctgatgatcggcctggcctggctggtttcctttgtgctctgggccccagccatcctcttctggcagtacctggtaggggagcggacagtgctagctgggcagtgctacatccagttcctctcccagcccatcatcacctttggcacagccatggctgccttctacctccctgtcacagtcatgtgcacgctctactggcgcatctaccgggagacagagaaccgagcacgggagctggcagcccttcagggctccgagacgccaggcaaagggggtggcagcagcagcagctcagagaggtctcagccaggggctgagggctcaccagagactcctccaggccgctgctgtcgctgctgccgggcccccaggctgctgcaggcctacagctggaaggaagaagaggaagaggacgaaggctccatggagtccctcacatcctcagagggagaggagcctggctccgaagtggtgatcaagatgccaatggtggaccccgaggcacaggcccccaccaagcagcccccacggagctccccaaatacagtcaagaggccgactaagaaagggcgtgatcgagctggcaagggccagaagccccgtggaaaggagcagctggccaagcggaagaccttctcgctggtcaaggagaagaaggcggctcggaccctgagtgccatcctcctggccttcatcctcacctggacaccgtacaacatcatggtgctggtgtccaccttctgcaaggactgtgttcccgagaccctgtgggagctgggctactggctgtgctacgtcaacagcaccatcaaccccatgtgctacgcactctgcaacaaagccttccgggacacctttcgcctgctgctgctttgccgctgggacaagagacgctggcgcaagatccccaagcgccctggctccgtgcaccgcactccctcccgccaatgctgatagtcccctctcctgcatccctccaccccagtccccgggaaaggccggtgggaagagggcaggggctgcatcctcagccccagggccctgctcaggcctcacctggcttcccaggaccctgggtcaccttcctgggcagcccagagagaccctgccaactttccagacttcgctattcccaggcagggagggaaacccggggaactggtttttctgttccctgctgggtgggaatgcgctcttcaccaggaagaaggcccgggaggaggatccgggctttggactccttgtttgcctttaggcaggaagtcaggagccagcagggcgggccaggagaaagaaggcttaacattaagtattccttggcccagcagcggcccagattgcggtgtgagatggtgccccctggggggcacagccagaaactgaactggccgctgggagaaaagccagatgacagggagctggggaatcccctcgcttcataggcagagcccgcccacctgggccctaggcatactctccaggattgtccacaaatgtcctcagagggtccctaggtgggtcaactccaaggcaaatgtccaagcatcagcaagacaatgacactggaagggtccggcttggctagtcacatatcaagtcccgaggcagcaacaggaccaggagccaggtgtcctgactgtcctacaatatcattttcctgggagtgggagtcaagtgtgcctgctatccagccgcaaatccataccccctgccccagagaagcctcagtccctccctcctggctcacagccaccacctggatggatctgctccatgcagatctagccaggcctcccgcatgctgcctgcctccggccctgccccacacaggcctggcccagccagcaggttctctcctgtgagctccccaatccaacccatgcatggcctcccagccacccggatctccaggcccagcctggccccaaatgttctttcctttcatcctcagcaagtgctgagtctgtgaataaagccacataaccagcgggcaaaaaaaaaaaaaaaaaaaaaaaaaaaaaaaaaaaaaaaaa

SEQ ID NO: 24 - DNA, 25 bp, Artificial Sequence
  Description of Artificial Sequence: Synthetic oligonucleotide
gcatcgacgccagcgccatcctacc

SEQ ID NO: 25 - DNA, 19 bp, Artificial Sequence
  Description of Artificial Sequence: Synthetic oligonucleotide
atgagctaggcgtcggcgg

SEQ ID NO: 26 - DNA, 24 bp, Artificial Sequence
  Description of Artificial Sequence: Synthetic oligonucleotide
tggaagaagaccgagtgtgtctac

SEQ ID NO: 27 - DNA, 24 bp, Artificial Sequence
  Description of Artificial Sequence: Synthetic oligonucleotide
ttctccctgggagagtaaggctgg

SEQ ID NO: 28 - DNA, 20 bp, Artificial Sequence
  Description of Artificial Sequence: Synthetic oligonucleotide
tctgctacaagttctggctt

SEQ ID NO: 29 - DNA, 20 bp, Artificial Sequence
  Description of Artificial Sequence: Synthetic oligonucleotide
ctgcagctttttctctaggg

SEQ ID NO: 30 - DNA, 21 bp, Artificial Sequence
  Description of Artificial Sequence: Synthetic oligonucleotide
aagctgcaaggtagcaacagc

SEQ ID NO: 31 - DNA, 21 bp, Artificial Sequence
  Description of Artificial Sequence: Synthetic oligonucleotide
aaccaacttatttcctaccac

SEQ ID NO: 32 - DNA, 20 bp, Artificial Sequence
  Description of Artificial Sequence: Synthetic oligonucleotide
cgaaggcaggtattttcaca

SEQ ID NO: 33 - DNA, 20 bp, Artificial Sequence
  Description of Artificial Sequence: Synthetic oligonucleotide
tggatctgatgctgggtttt

SEQ ID NO: 34 - DNA, 21 bp, Artificial Sequence
  Description of Artificial Sequence: Synthetic oligonucleotide
ttggcctattggtttgggaat

SEQ ID NO: 35 - DNA, 23 bp, Artificial Sequence
  Description of Artificial Sequence: Synthetic oligonucleotide
gtctgggaatttgaagcgtccac

SEQ ID NO: 36 - DNA, 2485 bp, Homo sapiens
tggggttggggaagtgcggtgactgggaaatgctgagctaggggcaggaggcatgggcgggacagtgttctgtgcccccttctagagcctaaatttgttgcccgagttcctccgggaagcgctccgggtagattctgggggccgggagctgagatccctgggcggggagctggggaagggatggggctgaggacagcgaagaagagggggcttgggggaggggggaaatggaagagagaggagggaggaggtacaagaggaaggagagaagtgcggccagcttgctttctccagtcgggtggccgcggggacccgggcgacgtcggaggccctgccgggaacccaggctgcagcccccacccccgcgctgcgacacgccccccaccccttccggctcacacccccgcccacactcctgagtggtgcggtgcagcgtcggccgaggcagcagagccgaggagagcaggtccacacctctgcatccctgcaccaggactcaccaagacgcccatcctggaaaaggtcccccgtaagatggcagcaaaaactcccagcagtgaggagtctgggctgcccaaactgcccgtgcccccgctgcagcagaccctggccacgtacctgcagtgcatgcgacacttggtgtctgaggagcagttcaggaagagccaggccattgtgcagcagtttggggcccctggtggcctcggcgagaccctgcagcagaaactcctggagcggcaggagaagacagccaactgggtgtctgagtactggctgaatgacatgtatctcaacaaccgcctggccctgcctgtcaactccagccctgccgtgatctttgctcggcagcacttccctggcaccgatgaccagctgaggtttgcagccagcctcatctctggtgtactcagctacaaggccctgctggacagccactccattcccactgactgtgccaaaggccagctgtcagggcagcccctttgcatgaagcaatactatgggctcttctcctcctaccggctccccggccatacccaggacacgctggtggctcagaacagcagcatcatgccggagcctgagcacgtcatcgtagcctgctgcaatcagttctttgtcttggatgttgtcattaatttccgccgtctcagtgagggggatctgttcactcagttgagaaagatagtcaaaatggcttccaacgaggacgagcgtttgcctccaattggcctgctgacgtctgacgggaggagcgagtgggccgaggccaggacggtcctcgtgaaagactccaccaaccgggactcgctggacatgattgagcgctgcatctgccttgtatgcctggacgcgccaggaggcgtggagctcagcgacacccacagggcactccagctccttcacggcggaggctacagcaagaacggggccaatcgctggtacgacaagtccctgcagtttgtggtgggccgagacggcacctgcggtgtggtgtgcgaacactccccattcgatggcatcgtcctggtgcagtgcactgagcatctgctcaagcacgtgacgcagagcagcaggaagctgatccgagcagactccgtcagcgagctccccgccccccggaggctgcggtggaaatgctccccggaaattcaaggccacttagcctcctcggcagaaaaacttcaacgaatagtaaagaaccttgacttcattgtctataagtttgacaactatgggaaaacattcattaagaagcagaaatgcagccctgatgccttcatccaggtggccctccagctggccttctacaggctccatcgaagactggtgcccacctacgagagcgcgtccatccgccgattccaggagggacgcgtggacaacatcagatcggccactccagaggcactggcttttgtgagagccgtgactgaccacaaggctgctgtgccagcttctgagaagcttctgctcctgaaggatgccatccgtgcccagactgcatacacagtcatggccataacagggatggccattgacaaccacctgctggcactgcgggagctggcccgggccatgtgcaaggagctgcccgagatgttcatggatgaaacctacctgatgagcaaccggtttgtcctctccactagccaggtgcccacaaccacggagatgttctgctgctatggtcctgtggtcccaaatgggtatggtgcctgctacaacccccagccagagaccatccttttctgcatctctagctttcacagctgcaaagagacttcttctagcaagtttgcaaaagctgtggaagaaagcctcattgacatgagagacctctgcagtctgctgccgcctactgagagcaagccattggcaacaaaggaaaaagccacgaggcccagccagggacaccaaccttgactcctgccactaggtttcacctcccaaacccagcctctagaacagccagaccctgcag

SEQ ID NO: 37 - DNA, 1199 bp, Homo sapiens
gcgcggaggccaggagctgagcggcggcggctgccggacgatgggagcgtgagcaggacggtgataacctctccccgatcgggttgcgagggcgccgggcagaggccaggacgcgagccgccagcggcgggacccatcgacgacttcccggggcgacaggagcagccccgagagccagggcgagcgcccgttccaggtggccggaccgcccgccgcgtccgcgccgcgctccctgcaggcaacgggagacgcccccgcgcagcgcgagcgcctcagcgcggccgctcgctctccccatcgagggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccagccttgcctccccgattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgagatcatcactggtatgccagcctcaactgaaggagcatatgtgtcttcagagtctcccattagaatatcagtatccacagaaggagcaaatacttcttcatctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccaga

SEQ ID NO: 38 - DNA, 20 bp, Artificial Sequence
  Description of Artificial Sequence: Synthetic oligonucleotide
tttcagaggaggccaagaga

SEQ ID NO: 39 - DNA, 20 bp, Artificial Sequence
  Description of Artificial Sequence: Synthetic oligonucleotide
ttcaggaggtctggggtaga

SEQ ID NO: 40-41 - skipped (2 entries)

SEQ ID NO: 42 - DNA, 20 bp, Artificial Sequence
  Description of Artificial Sequence: Synthetic oligonucleotide
gtgagaaagccccaggttac

SEQ ID NO: 43 - DNA, 21 bp, Artificial Sequence
  Description of Artificial Sequence: Synthetic oligonucleotide
ccaggctggtctggacttctg

SEQ ID NO: 44 - DNA, 26 bp, Artificial Sequence
  Description of Artificial Sequence: Synthetic oligonucleotide
aaggcatcagttttcaatagcttttt

SEQ ID NO: 45 - DNA, 27 bp, Artificial Sequence
  Description of Artificial Sequence: Synthetic oligonucleotide
taagtagaaatgggaactctccatctc

SEQ ID NO: 46 - DNA, 17 bp, Artificial Sequence
  Description of Artificial Sequence: Synthetic oligonucleotide
tttattttgccaaatat

SEQ ID NO: 47 - DNA, 22 bp, Artificial Sequence
  Description of Artificial Sequence: Synthetic oligonucleotide
tctttattttaccaaatatcat

SEQ ID NO: 48-51 - skipped (4 entries)

SEQ ID NO: 52 - DNA, 52 bp, Artificial Sequence
  Description of Artificial Sequence: Synthetic oligonucleotide
cgagaacggaggtagctttttaaaaasgaagacacactcggtcttcttccat

SEQ ID NO: 53 - DNA, 52 bp, Artificial Sequence
  Description of Artificial Sequence: Synthetic oligonucleotide
tatgtcctcggagtgctgtgagtgtcyggcacttccatccaaagccaacagt

SEQ ID NO: 54 - DNA, 52 bp, Artificial Sequence
  Description of Artificial Sequence: Synthetic oligonucleotide
cctaattggcctattggtttggcaatstgatatttctgtgagcccagtagca

SEQ ID NO: 55 - DNA, 52 bp, Artificial Sequence
  Description of Artificial Sequence: Synthetic oligonucleotide
ccaaatgtagccacacatcatagtcayctagattcctgggtctaccccagac

SEQ ID NO: 56 - DNA, 52 bp, Artificial Sequence
  Description of Artificial Sequence: Synthetic oligonucleotide
agaatgtcttgagaatccagtgtctcygcagaaagcagtcttccaaacatgc

SEQ ID NO: 57 - DNA, 52 bp, Artificial Sequence
  Description of Artificial Sequence: Synthetic oligonucleotide
acatatgaattttggggagaacacaarcattcagacaatagcatatacatat

SEQ ID NO: 58 - DNA, 51 bp, Artificial Sequence
  Description of Artificial Sequence: Synthetic oligonucleotide
cccttacccgcatctcccacccccargacgcccctttcgccccaacggtct

SEQ ID NO: 59 - DNA, 51 bp, Artificial Sequence
  Description of Artificial Sequence: Synthetic oligonucleotide
tggacgtccagctgggcgcctgcctygaccagcactttgaggatggctgtg

SEQ ID NO: 60 - DNA, 52 bp, Artificial Sequence; identical to SEQ ID NO: 52
  Description of Artificial Sequence: Synthetic oligonucleotide

SEQ ID NO: 61 - DNA, 52 bp, Artificial Sequence
  Description of Artificial Sequence: Synthetic oligonucleotide
aggctctacagtaatgactttaactcyggagaagctaacacttctgatgcat

SEQ ID NO: 62 - DNA, 52 bp, Artificial Sequence; identical to SEQ ID NO: 56
  Description of Artificial Sequence: Synthetic oligonucleotide

SEQ ID NO: 63 - DNA, 51 bp, Artificial Sequence; identical to SEQ ID NO: 58
  Description of Artificial Sequence: Synthetic oligonucleotide

SEQ ID NO: 64 - DNA, 51 bp, Artificial Sequence; identical to SEQ ID NO: 59
  Description of Artificial Sequence: Synthetic oligonucleotide

SEQ ID NO: 65 - DNA, 49 bp, Artificial Sequence
  Description of Artificial Sequence: Synthetic oligonucleotide
ggcagggggagcgggcgtggagggygcgcacgaggtcgaggcgagtccg

SEQ ID NO: 66 - DNA, 51 bp, Artificial Sequence
  Description of Artificial Sequence: Synthetic oligonucleotide
cccagcggatggtggatttcgctggcrtgaaggacaaggtgtgcatgcctg

SEQ ID NO: 67 - DNA, 51 bp, Artificial Sequence; identical to SEQ ID NO: 59
  Description of Artificial Sequence: Synthetic oligonucleotide

SEQ ID NO: 68 - DNA, 49 bp, Artificial Sequence; identical to SEQ ID NO: 65
  Description of Artificial Sequence: Synthetic oligonucleotide

SEQ ID NO: 69 - DNA, 52 bp, Artificial Sequence
  Description of Artificial Sequence: Synthetic oligonucleotide
ttttttttttttttcttatttaccacmggacataaatgcaaggaattttgat

SEQ ID NO: 70 - DNA, 52 bp, Artificial Sequence; identical to SEQ ID NO: 69
  Description of Artificial Sequence: Synthetic oligonucleotide

SEQ ID NO: 71 - DNA, 51 bp, Artificial Sequence
  Description of Artificial Sequence: Synthetic oligonucleotide
ctggggggccgtttgccctagagatgygggtcctgcaccgcctctgtttgg

SEQ ID NO: 72 - DNA, 51 bp, Artificial Sequence
  Description of Artificial Sequence: Synthetic oligonucleotide
caccagagatgtggccggaatgcagaratgaagcactgagcacagtaggta

SEQ ID NO: 73 - DNA, 52 bp, Artificial Sequence
  Description of Artificial Sequence: Synthetic oligonucleotide
actaaaaaagagatatatgatatttggyaaaataaagatacatggcttccag

SEQ ID NO: 74 - DNA, 52 bp, Artificial Sequence; identical to SEQ ID NO: 73
  Description of Artificial Sequence: Synthetic oligonucleotide

SEQ ID NO: 75 - DNA, 52 bp, Artificial Sequence; identical to SEQ ID NO: 61
  Description of Artificial Sequence: Synthetic oligonucleotide

SEQ ID NO: 76 - DNA, 51 bp, Artificial Sequence; identical to SEQ ID NO: 72
  Description of Artificial Sequence: Synthetic oligonucleotide